SEQUENCE LISTING (13 sequences)

SEQ ID NO: 1 - DNA, 100 bp, Artificial Sequence
  NanoString probe - ActB
ccgccgagaccgcgtccgccccgcgagcacagagcctcgcctttgccgatccgccgcccgtccacacccgccgccagctcaccatggatgatgatatcgc

SEQ ID NO: 2 - DNA, 100 bp, Artificial Sequence
  NanoString probe - AKR1C3
ggtgacgcagaggacgtctctatgccggtgactggacatatcacctctacttaaatccgtcctgtttagcgacttcagtcaactacagctgagtccatag

SEQ ID NO: 3 - DNA, 100 bp, Artificial Sequence
  NanoString probe - B2M
ccaagatagttaagtgggatcgagacatgtaagcagcatcatggaggtttgaagatgccgcatttggattggatgaattccaaattctgcttgcttgctt

SEQ ID NO: 4 - DNA, 100 bp, Artificial Sequence
  NanoString probe - CD40
tgcatgcagagaaaaacagtacctaataaacagtcagtgctgttctttgtgccagccaggacagaaactggtgagtgactgcacagagttcactgaaacg

SEQ ID NO: 5 - DNA, 100 bp, Artificial Sequence
  NanoString probe - CTLA4
aggcatcgccagctttgtgtgtgagtatgcatctccaggcaaagccactgaggtccgggtgacagtgcttcggcaggctgacagccaggtgactgaagtc

SEQ ID NO: 6 - DNA, 100 bp, Artificial Sequence
  NanoString probe - GAPDH
acatgttccaatatgattccacccatggcaaattccatggcaccgtcaaggctgagaacgggaagcttgtcatcaatggaaatcccatcaccatcttcca

SEQ ID NO: 7 - DNA, 100 bp, Artificial Sequence
  NanoString probe - HPRT1
gatgatctctcaactttaactggaaagaatgtcttgattgtggaagatataattgacactggcaaaacaatgcagactttgctttccttggtcaggcagt

SEQ ID NO: 8 - DNA, 100 bp, Artificial Sequence
  NanoString probe - ID3
aacgcaggtgctggcgcccgttctgcctgggaccccgggaacctctcctgccggaagccggacggcagggatgggccccaacttcgccctgcccacttga

SEQ ID NO: 9 - DNA, 100 bp, Artificial Sequence
  NanoString probe - MS4A1
cttctgatgatcccagcagggatctatgcacccatctgtgtgactgtgtggtaccctctctggggaggcattatgtatattatttccggatcactcctgg

SEQ ID NO: 10 - DNA, 100 bp, Artificial Sequence
  NanoString probe - MZB1
gcaaaatctggcaaaggcagagaccaaacttcatacctcaaactctggggggcggcgggagctgagcgagttggtctacacggatgtcctggaccggagc

SEQ ID NO: 11 - DNA, 100 bp, Artificial Sequence
  NanoString probe - TCL1A
agcacgcctggctgcccttaaccatcgagataaaggataggttacagttacgggtgctcttgcgtcgggaagacgtcgtcctggggaggcctatgacccc

SEQ ID NO: 12 - DNA, 100 bp, Artificial Sequence
  NanoString probe - TUBA4A
tgtgaaactggtgctggaaaacacgtaccccgggcagtttttgtggatctggagcctacggtcattgatgagatccgaaatggcccataccgacagctct

SEQ ID NO: 13 - DNA, 100 bp, Artificial Sequence
  NanoString probe - YWHAZ
cttggtggccatgtacttggaaaaaggccgcatgatctttctggctccactcagtgtctaaggcaccctgcttcctttgcttgcatcccacagactattt

(19) United States
(12) Patent Application Publication
(10) Pub. No.: US 2024/0240254 A1
(43) Pub. Date: Jul. 18, 2024

(54) NON-INVASIVE DIAGNOSIS OF SUBCLINICAL REJECTION

(71) Applicants: CENTRE HOSPITALIER UNIVERSITAIRE DE NANTES, Nantes CEDEX 1 (FR); NANTES UNIVERSITÉ, Nantes (FR); INSERM (INSTITUT NATIONAL DE LA SANTÉ ET DE LA RECHERCHE MÉDICALE), Paris CEDEX 13 (FR)

(72) Inventors: Sophie BROUARD, Nantes (FR); Richard DANGER, Nantes (FR); Magali GIRAL, Tourville (FR)

(73) Assignees: CENTRE HOSPITALIER UNIVERSITAIRE DE NANTES, Nantes CEDEX 1 (FR); NANTES UNIVERSITÉ, Nantes (FR); INSERM (INSTITUT NATIONAL DE LA SANTÉ ET DE LA RECHERCHE MÉDICALE), Paris CEDEX 13 (FR)

(21) Appl. No.: 18/564,834

(22) PCT Filed: May 25, 2022
(86) PCT No.: PCT/EP2022/064247

§ 371 Date: Nov. 28, 2023

(30) Foreign Application Priority Data
May 28, 2021 (EP) 21305713.6

(51) Int. Cl. C12Q 1/6883

(52) U.S. Cl. CPC C12Q 1/6883; C12Q 2600/158

(57) ABSTRACT

Methods of diagnosing subclinical rejection, based on the level, amount or concentration of two genes, TCL1A and AKR1C3, whether independently from each other or in combination. When in combination, the level, amount or concentration of the two genes can further be combined with clinical parameters in the form of a composite score. Also, methods for treating subclinical rejection in a subject if/when the subject was diagnosed with subclinical rejection using the method of the invention; as well as a computer system and a kit-of-parts for implementing the method of diagnosing subclinical rejection.

### FIELD OF INVENTION

The invention relates to the field of subclinical rejection (SCR), and provides means and methods for diagnosing SCR in a routine manner, using non-invasive markers.

### BACKGROUND OF INVENTION

In kidney transplantation, subclinical rejection (SCR), and in particular antibody-mediated subclinical rejection (sABMR), is a major threat associated with unfavorable allograft outcomes (Filippone & Farber, 2020. Transplantation; Loupy et al., 2015. J Am Soc Nephrol. 26(7): 1721-31; Mehta et al., 2016. Transplantation. 100(8): 1610-8; Rush & Gibson, 2019. Transplantation. 103(6):e139-e145; Shishido et al., 2003. J Am Soc Nephrol. 14(4): 1046-52).

Nevertheless, while acute and chronic rejection diagnosis can be clinically suspected from allograft dysfunction and confirmed by histology, SCR is, by definition, associated with stable graft function while graft lesions are established. Its diagnosis can therefore not rely on traditional kidney function measurements, like serum creatinine or glomerular filtration rates. Surveillance biopsies within the first year of follow-up have thus been proposed in routine practice to diagnose and prevent or ultimately treat early subclinical lesions (Hoffman et al., 2019. Transplantation. 103(7): 1457-1467; Loupy et al., 2015. J Am Soc Nephrol. 26(7): 1721-31; Moreso et al., 2004. Transplantation. 78(7):1064-8; Nankivell et al., 2004. Transplantation. 78(2):242-9; Rush et al., 1998. J Am Soc Nephrol. 9(11):2129-34). But graft biopsy is an at-risk intervention (Fereira et al., 2004. Transplantation. 77(9): 1475-6) that is not performed in all transplant centers (Couvrat-Desvergnes et al., 2019. Nephrol Dial Transplant. 34(4): 703-711; Mehta et al., 2017. Clin Transplant. 31(5)). Organ biopsy results can also be inaccurate, in particular if the area biopsied is not representative of the health of the organ as a whole.

When one-year surveillance biopsies are performed, half display normal or subnormal histology and SCR represents only 25% of the cases (Couvrat-Desvergnes et al., 2019. Nephrol Dial Transplant. 34(4): 703-711; Loupy et al., 2015. J Am Soc Nephrol. 26(7): 1721-31; Nankivell et al., 2004. Transplantation. 78(2):242-9).

There is thus a need for non-invasive biomarkers in absence of graft function decline, not only to detect early SCR, but also to avoid invasive procedures on the majority of patients with no serious histological lesions. Irrespective of center biopsy habits, such non-invasive biomarkers could be used as a screening tool to guide surveillance biopsy needs and to improve patient management (Friedewald & Abecassis, 2019. Am J Transplant. 19(7): 2141-2142).

Several biomarkers of SCR have previously been proposed, including blood gene signatures (WO 2015/179777; WO 2019/217910; Crespo et al., 2017. Transplantation. 101(6):1400-1409; Friedewald et al., 2019. Am J Transplant. 19(1):98-109; Van Loon et al., 2019. EBioMedicine. 46:463-472; Zhang et al., 2019. J Am Soc Nephrol. 30(8): 1481-1494).

Zhang published a signature of 17 genes able to diagnose SCR and acute cellular rejection with an 89% negative predictive value and a 73% positive predictive value at 3-month post-transplantation (Zhang et al., 2019. J Am Soc Nephrol. 30(8):1481-1494). Similarly, a signature of 51 genes allows identifying SCR 24-month post-transplantation (Friedewald et al., 2019. Am J Transplant. 19(1):98-109). However, both these studies focus on cellular and borderline rejections only. Van Loon reported on a signature of 8 genes to diagnose only antibody-mediated rejection (ABMR) (Van Loon et al., 2019. EBioMedicine. 46:463-472). Finally, the 17-gene signature of the kSort study has also been proposed to diagnose 6-month subclinical ABMR (sABMR) (Crespo et al., 2017. Transplantation. 101(6): 1400-1409) but was not validated in a large cohort of 1134 patients (Van Loon et al., 2021. Am J Transplant. 21(2):740-750). Thus, none of these signatures are currently routinely used yet.

Hence, there still remains a need for non-invasive biomarkers capable for detecting SCR, in a routine manner.

Here, the Inventors show that two genes, TCL1A and AKR1C3, allow, independently from each other, to identify patients affected with SCR; and that the combination of both these genes allows an even better discrimination. The Inventors further propose a composite score based on the expression of TCL1A and AKR1C3, combined with three clinical variables (the experience of rejection episodes before blood sampling, the gender of the graft recipient and the uptake of immunosuppressant, whether cyclosporine A [CsA] or tacrolimus, at blood sampling time) to identify SCR-free patients at one-year post transplantation.

### SUMMARY

The present invention relates to a method of diagnosing subclinical kidney rejection in a subject in need thereof, comprising the steps of:
a) determining the level, amount or concentration of at least one biomarker selected from the group consisting of TCL1A and AKR1C3 in a sample previously taken from the subject;
b) comparing the level, amount or concentration of the at least one biomarker with the level, amount or concentration of the same at least one biomarker determined in at least one reference subject,
wherein the at least one reference subject is:
   a subject who has not undergone kidney transplantation,
   a kidney transplant recipient who is not affected with subclinical kidney rejection, or
   the subject investigated for subclinical kidney rejection themselves prior to kidney transplantation; and
c) concluding that the subject is affected with subclinical kidney rejection when the level, amount or concentration of the at least one biomarker is statistically significantly lower than the level, amount or concentration of the same at least one biomarker determined in the at least one reference subject.

In some embodiments, step a) does not comprise determining the level, amount or concentration of CD40, CTLA4, ID3, and/or MZB1. In some embodiments, step a) does not comprise determining the level, amount or concentration of a biomarker other than TCL1A and/or AKR1C3.

In some embodiments, step a) comprises determining the level, amount or concentration of TCL1A in the sample previously taken from the subject. In some embodiments, step a) comprises determining the level, amount or concentration of AKR1C3 in the sample previously taken from the subject. In some embodiments, step a) comprises determining the level, amount or concentration of both TCL1A and AKR1C3 in the sample previously taken from the subject.

In some embodiments, the level, amount or concentration of the at least one biomarker is expressed in terms of absolute or relative levels, amounts or concentrations; preferably is expressed in terms of relative levels, amounts or concentrations normalized relative to the level, amount or concentration of one or several reference markers.

In some embodiments, the method comprises:
a) determining a composite score with the level, amount or concentration of the at least one biomarker selected from the group consisting of TCL1A and AKR1C3, preferably of both TCL1A and AKR1C3, wherein said composite score is established using Formula (1):

$$composite score = \sum {β}_{i} {X}_{i} + {β}_{0} \qquad ( 1 )$$

wherein:
“βᵢ” represents the regression coefficient for the level, amount or concentration of each of the at least one biomarker;
“Xᵢ” represent the predictor variable for the level, amount or concentration of each of the at least one biomarker;
“β₀” represents the intercept of the equation,
b) comparing the composite score with a reference composite score determined in the at least one reference subject;
c) concluding that the subject is affected with subclinical kidney rejection when the composite score is substantially higher than the reference composite score determined in the at least one reference subject.

In some embodiments, the method comprises:
a) determining a composite score, with:
   the level, amount or concentration of the at least one biomarker selected from the group consisting of TCL1A and AKR1C3, preferably of both TCL1A and AKR1C3; and
   one, two, or preferably three clinical parameters selected among:
      the experience of rejection episodes before blood sampling,
      the recipient gender, and
      the uptake of immunosuppressant (IS) at blood sampling, preferably the uptake of tacrolimus or of cyclosporine A (CsA) at blood sampling,
wherein said composite score is established using Formula (2):

$$composite score = \sum ( {β}_{previous rejection episode} \times previous rejection episode + {β}_{IS uptake} \times IS uptake + {β}_{recipient gender} \times recipient gender + {β}_{TCL 1 A} \times Expr ( TCL 1 A ) + {β}_{AKR 1 C 3} \times Expr ( AKR 1 C 3 ) ) + {β}_{0} \qquad ( 2 )$$

wherein:
“β_{TCL1A}”, “β_{AKR1C3}”, “β_{previous rejection episode}”, “β_{IS uptake}”, and “β_{recipient gender}” represent the regression coefficients for each predictor among the level, amount or concentration of the biomarkers and the clinical parameters;
“previous rejection episode” represents the predictor variable defining the experience of rejection episodes before blood sampling, with 0=“no previous rejection episodes” and 1=“one or several previous rejection episodes”;
“IS uptake” represents the predictor variable defining the uptake of immunosuppressant (IS) at blood sampling time, preferably of tacrolimus or of cyclosporine A (CsA), with 0=“no CsA uptake” or “tacrolimus uptake”, and 1=“CsA uptake” or “no tacrolimus uptake”;
“recipient gender” represents the predictor variable defining the gender of the graft recipient, with 0=“female” and 1=“male”;
“Expr(TCL1A)” and “Expr(AKR1C3)” represent the predictor variables defining the level, amount or concentration of TCL1A and AKR1C3, respectively;
“β₀” represents the intercept of the equation;
b) comparing the composite score with a reference composite score determined in the at least one reference subject;
c) concluding that the subject is affected with subclinical kidney rejection when the composite score is substantially higher than the reference composite score determined in the at least one reference subject.

In this embodiment, the uptake of immunosuppressant (IS) at blood sampling may be the uptake of tacrolimus at blood sampling, or the uptake of cyclosporine A (CsA) at blood sampling.

In this embodiment, the composite score may be determined with:
the level, amount or concentration of both TCL1A and AKR1C3, and
the three following clinical parameters: (i) the experience of rejection episodes before blood sampling, (ii) the recipient gender and (iii) the uptake of cyclosporine A (CsA) at blood sampling.

In this embodiment, subclinical kidney rejection is subclinical T-cell mediated kidney rejection (sTCMR), subclinical antibody-mediated kidney rejection (sABMR), and/or mixed sTCMR/sABMR.

In some embodiments, the method comprises:
a) determining a composite score, with:
   the level, amount or concentration of the at least one biomarker selected from the group consisting of TCL1A and AKR1C3, preferably of both TCL1A and AKR1C3; and
   one, two, three or preferably four clinical parameters selected among:
      the experience of rejection episodes before blood sampling,
      the recipient gender,
      the allograft rank, and
      the number of donor-recipient HLA mismatches,
wherein said composite score is established using Formula (3):

$$composite score = \sum ( {β}_{previous rejection episode} \times previous rejection episode + {β}_{allograft rank} \times allograft rank + {β}_{HLA mismatches} \times HLA mismatches + {β}_{recipient gender} \times recipient gender + {β}_{TCL 1 A} \times Expr ( TCL 1 A ) + {β}_{AKR 1 C 3} \times Expr ( AKR 1 C 3 ) ) + {β}_{0} \qquad ( 3 )$$

wherein:
“β_{TCL1A}”, “β_{AKR1C3}”, “β_{previous rejection episode}”, “β_{allograft rank}”, “β_{HLA mismatches}”, and “β_{recipient gender}” represent the regression coefficients for each predictor among the level, amount or concentration of the biomarkers and the clinical parameters;
“previous rejection episode” represents the predictor variable defining the experience of rejection episodes before blood sampling, with 0=“no previous rejection episodes” and 1=“one or several previous rejection episodes”;
“allograft rank” represents the predictor variable defining the occurrence of previous transplantation, with 0=“no previous transplantation” and 1=“one or several previous transplantations”;
“HLA mismatches” represents the predictor variable defining the occurrence of donor-recipient HLA mismatches, with 0=“3 or less HLA-A, -B and/or -DR mismatches”, and 1=“strictly more than 3 HLA-A, -B and/or -DR mismatches”;
“recipient gender” represents the predictor variable defining the gender of the graft recipient, with 0=“female” and 1=“male”;
“Expr(TCL1A)” and “Expr(AKR1C3)” represent the predictor variables defining the level, amount or concentration of TCL1A and AKR1C3, respectively;
“β₀” represents the intercept of the equation;
b) comparing the composite score with a reference composite score determined in the at least one reference subject;
c) concluding that the subject is affected with subclinical kidney rejection when the composite score is substantially higher than the reference composite score determined in the at least one reference subject.

In this embodiment, subclinical kidney rejection consists of subclinical antibody-mediated kidney rejection (sABMR).

In some embodiments, the at least one reference subject is a reference population comprising two or more reference subjects.

In some embodiments, the method is computed-implemented.

The present invention also relates to a computer system for diagnosing subclinical kidney rejection in a subject in need thereof, the computer system comprising:
i) at least one processor, and
ii) at least one storage medium that stores at least one code readable by the processor, and which, when executed by the processor, causes the processor to:
   a. receive an input level, amount or concentration of the at least one biomarker selected from the group consisting of TCL1A and AKR1C3,
   b. analyze and transform the input level, amount or concentration to derive a composite score established using Formula (1) as defined in claim 8,
   c. generate an output, wherein the output is the composite score, and
   d. provide a diagnosis of the subject as being affected or not with subclinical kidney rejection based on the output.

In some embodiments, the at least one code readable by the processor, when executed by the processor, causes the processor to:
a. receive input levels, amounts or concentrations of the at least one biomarker selected from the group consisting of TCL1A and AKR1C3, and input values for one, two, or preferably three clinical parameters selected among
   (i) the experience of rejection episodes before blood sampling,
   (ii) the recipient gender and
   (iii) the uptake of immunosuppressant (IS) at blood sampling, preferably the uptake of tacrolimus or of cyclosporine A (CsA) at blood sampling,
b. analyze and transform the input levels, amounts or concentrations, and the input values, to derive a composite score established using Formula (2) as defined in claim 9,
c. generate an output, wherein the output is the composite score, and
d. provide a diagnosis of the subject as being affected or not with subclinical kidney rejection based on the output.

In this embodiment, the uptake of immunosuppressant (IS) at blood sampling may be the uptake of tacrolimus at blood sampling, or the uptake of cyclosporine A (CsA) at blood sampling.

In this embodiment, subclinical kidney rejection is subclinical T-cell mediated kidney rejection (sTCMR), subclinical antibody-mediated kidney rejection (sABMR), and/or mixed sTCMR/sABMR.

In some embodiments, the at least one code readable by the processor, when executed by the processor, causes the processor to:
a. receive input levels, amounts or concentrations of the at least one biomarker selected from the group consisting of TCL1A and AKR1C3, and input values for one, two, three or preferably four clinical parameters selected among
   (i) the experience of rejection episodes before blood sampling,
   (ii) the recipient gender,
   (iii) previous transplantation, and
   (iv) the number of donor-recipient HLA mismatches,
b. analyze and transform the input levels, amounts or concentrations, and the input values, to derive a composite score established using Formula (3) as defined in claim 14,
c. generate an output, wherein the output is the composite score, and
d. provide a diagnosis of the subject as being affected or not with subclinical kidney rejection based on the output.

In this embodiment, subclinical kidney rejection consists of subclinical antibody-mediated kidney rejection (sABMR).

In some embodiments, the subject is diagnosed as being affected with subclinical kidney rejection when the output is substantially higher than the same output obtained in at least one reference subject, wherein the reference subject is a subject who has not undergone kidney transplantation, a kidney transplant recipient who is not affected with subclinical rejection, or the subject investigated for subclinical rejection themselves prior to kidney transplantation.

The present invention also relates to a computer program comprising software code readable by a processor adapted to perform, when executed by said processor, the computer-implemented method of diagnosing subclinical kidney rejection disclosed herein.

The present invention also relates to a non-transitory computer-readable storage medium comprising code which, when executed by a computer, causes a processor to carry out the computer-implemented method of diagnosing subclinical kidney rejection disclosed herein.

The present invention also relates to a kit-of-parts for performing the method of diagnosing subclinical kidney rejection disclosed herein, comprising means for determining the level, amount or concentration of at least one biomarker selected from the group consisting of TCL1A and AKR1C3, and optionally, means for determining the level, amount or concentration of at least one reference marker and instructions for use to perform the method.

In some embodiments, said means are selected from the group consisting of nucleic acid probes, antibodies, and aptamers.

### Definitions

In the present invention, the following terms have the following meanings.

“AKR1C3” refers to the gene encoding the aldo-keto reductase family 1 member C3 protein. The naturally occurring human AKR1C3 gene has a nucleotide sequence as shown in Genbank Accession number NM_001253908 (version 2 of May 9, 2021) and the naturally occurring human aldo-keto reductase family 1 member C3 protein has an amino acid sequence as shown in Genbank Accession number NP_001240837 (version 1 of May 9, 2021) or in UniProt Accession number P42330 (version 4 of Oct. 5, 2010).

“TCL1A” refers to gene encoding the T-cell leukemia/lymphoma protein 1A. The naturally occurring human TCL1A gene has a nucleotide sequence as shown in Genbank Accession number NM_001098725 (version 2 of Apr. 18, 2021) and the naturally occurring human T-cell leukemia/lymphoma protein 1A has an amino acid sequence as shown in Genbank Accession number NP_001092195 (version 1 of Apr. 18, 2021) or in UniProt Accession number P56279 (version 1 of Jul. 15, 1998).

“Biological sample” refers to any sample obtained from a subject, preferably from a transplanted subject, such as a blood sample, a serum sample, a plasma sample, a urine sample, a lymph sample, or a biopsy.

“Immunosuppressive therapy” or “immunosuppressive treatment” refer to the administration to a transplanted subject of one or more immunosuppressive drugs (or immunosuppressant). Immunosuppressive drugs that may be employed in transplantation procedures include all those described in the therapeutic subgroup L04 of the Anatomical Therapeutic Chemical Classification System (ATC/DDD Index 2021) developed by the World Health Organization (WHO) for the classification of drugs and other medical products, which are hereby incorporated by reference. Further examples include, but are not limited to, purine synthesis inhibitors (such as azathioprine, mycophenolic acid, mycophenolate mofetil), pyrimidine synthesis inhibitors (such as leflunomide, teriflunomide), antifolate (such as methotrexate), tacrolimus, ciclosporin, pimecrolimus, voclosporin, abetimus, gusperimus, immunomodulatory imide drugs (such as lenalidomide, pomalidomide, thalidomide, apremilast), IL-1 receptor antagonists (such as anakinra), mTOR inhibitors (such as sirolimus, everolimus, ridaforolimus, temsirolimus, umirolimus, zotarolimus), anti-complement component 5 antibodies (such as eculizumab), anti-TNF antibodies (such as adalimumab, afelimomab, certolizumab pegol, golimumab, infliximab, nerelimomab), TNF inhibitors (such as etanercept, pegsunercept), anti-interleukin-5 antibodies (such as mepolizumab), VEGF inhibitors (such as aflibercept), anti-immunoglobulin E antibodies (such as omalizumab), anti-interferon antibodies (such as faralimomab), anti-interleukin-6 antibodies (such as clazakizumab, elsilimomab, filgotinib), anti-interleukin-12 and/or interleukin-23 antibodies (such as lebrikizumab, ustekinumab), anti-interleukin-17A antibodies (such as secukinumab), interleukin-1 inhibitors (such as rilonacept), anti-CD3 antibodies (such as muromonab-CD3, otelixizumab, teplizumab, visilizumab), anti-CD4 antibodies (such as clenoliximab, keliximab, zanolimumab), anti-CD11a antibodies (such as efalizumab), anti-CD18 antibodies (such as erlizumab), anti-CD20 antibodies (such as obinutuzumab, rituximab, ocrelizumab, pascolizumab), anti-CD23 antibodies (such as gomiliximab, lumiliximab), anti-CD40 antibodies (such as teneliximab, toralizumab), anti-CD62L/L-selectin antibodies (such as aselizumab), anti-CD80 antibodies (such as galiximab), anti-CD147 antibodies (such as gavilimomab), anti-CD154 antibodies (such as ruplizumab), anti-BLyS antibodies (such as belimumab, blisibimod), anti-CTLA-4 antibodies (such as abatacept), CTLA-4 fusion proteins (such as abatacept, belatacept), anti-CAT antibodies (such as bertilimumab, lerdelimumab, metelimumab), anti-integrin antibodies (such as natalizumab, vedolizumab), anti-interleukin-6 receptor antibodies (such as tocilizumab), anti-LFA-1 antibodies (such as odulimomab), anti-interleukin-2 receptor antibodies (such as basiliximab, daclizumab, inolimomab), anti-CD5 antibodies (such as zolimomab aritox), polyclonal antibody infusions (such as anti-thymocyte globulin, anti-lymphocyte globulin), and other monoclonal antibodies such as atorolimumab, cedelizumab, fontolizumab, maslimomab, morolimumab, pexelizumab, reslizumab, rovelizumab, siplizumab, talizumab, telimomab aritox, vapaliximab, vepalimomab. These drugs may be used in monotherapy or in combination therapies.

“Organ transplantation” refers to the procedure of replacing diseased organs, parts of organs, or tissues by healthy organs or tissues. The transplanted organ or tissue can be obtained either from the subject himself (it is then referred to as an “autograft”), from another human donor (it is then referred to as an “allograft”) or from an animal (it is then referred to as an “xenograft”). Transplanted organs may be artificial or natural, whole (such as kidney, heart and liver) or partial (such as heart valves, skin and bone).

“Subclinical (kidney) rejection” or “SCR” refers to histologically-defined lesions of a kidney graft according the Banff classification typically identified by surveillance biopsies during post-transplantation follow-ups (an at-risk intervention, that is not performed in all transplant centers), but without concurrent functional deterioration of the kidney graft (variably defined as a serum creatinine level not exceeding 10%, 20% or 25% of baseline values, i.e., ranging from about 0.6 to about 1.2 mg/dL in adult males and from about 0.5 to 1.1 mg/dL in adult females, although kidney transplant recipients typically have serum creatinine levels ranging from about 1.0 to about 1.9 mg/dL in adult males and from about 0.8 to about 1.5 mg/dL in female subjects). It is clinically distinct from acute or chronic rejection, which is characterized by functional renal impairment measured by an elevated serum creatinine exceeding 10%, 20% or 25% of baseline values as defined above. SCR can be divided into two categories: one is primarily a cellular response by way of cytotoxic T lymphocytes that have become specifically activated against donor antigens and which then directly infiltrate, attack and injure the engrafted organ: “subclinical T-cell mediated rejection” or “sTCMR”. The other is a humoral response, wherein the organ recipient's immune system produces donor-specific antibodies (DSA) to the donor organ, leading to an immune assault upon and injury to the engrafted organ: “subclinical antibody-mediated rejection” or “sABMR”. These two immune mechanisms may also coexist and are then referred to as “mixed sTCMR and SABMR” or “mixed SCR”.

“Subject” refers to any mammals including, but are not limited to, humans, non-human primates (such as, e.g., chimpanzees, and other apes and monkey species), farm animals (such as, e.g., cattle, horses, sheep, goats, and swine), domestic animals (such as, e.g., rabbits, dogs, and cats), laboratory animals (such as, e.g., rats, mice and guinea pigs), and the like. The term does not denote a particular age or gender, unless explicitly stated otherwise. In particular, the subject is a human, also termed “patient”. In a particular embodiment, the subject is a transplanted subject, also termed “graft or transplant recipient” or “grafted or transplanted subject”.

“Transplanted subject” (or “graft or transplant recipient” or “grafted subject”) refers to a subject who has received an organ transplantation.

### DETAILED DESCRIPTION

This invention relates to a method of diagnosing subclinical rejection in a subject in need thereof.

The term “diagnosis” and its declensions refer to an estimation or a determination of whether or not a subject is suffering from a given disease or condition, or of estimating or determining the severity of the given disease or condition, e.g., subclinical rejection. Diagnosis does not require ability to determine the presence or absence of a particular disease with 100% accuracy, or even that a given course or outcome is more likely to occur than not. Instead, the “diagnosis” refers to an increased probability that a subject is affected with a certain disease or condition as compared to the probability that this subject is not affected with this certain disease or condition.

In one embodiment, the subject is a mammal. In a particular embodiment, the subject is a human.

In one embodiment, the subject is a transplanted subject. In a particular embodiment, the subject is a kidney transplant recipient. In one embodiment, the kidney transplant recipient may further have been grafted with the pancreas and/or a piece of duodenum of the kidney donor.

In one embodiment, the subject was grafted with the kidney about 1 month, 2 months, 3 months, 6 months, 9 months or 1 year prior to performing the method of the invention.

In one embodiment, the subject is under immunosuppressive therapy, i.e., the subject is administered with one or more immunosuppressive drugs.

In one embodiment, the subject does not display functional deterioration of the kidney graft. In one embodiment, the subject's serum creatinine levels are below 3 mg/dL, below 2.5 mg/dL, below 2 mg/dL. In one embodiment, the subject's serum creatinine levels range from about 0.5 to about 2.0 mg/dL.

In one embodiment, the subject is not affected with acute rejection.

In one embodiment, the subject is an operationally tolerant kidney transplant recipient. In one embodiment, the subject is a non-operationally tolerant kidney transplant recipient. Means and methods of determining whether a kidney transplant recipient is operationally tolerant or not have been described in the art, in particular in WO 2018/015551 or in Danger et al., 2017 (Kidney Int. 91(6): 1473-1481).

In one embodiment, the subject is at risk of subclinical rejection. Examples of risk factors for subclinical rejection include, but are not limited to, immunosuppressive therapy, prior acute rejection, chronic allograft nephropathy (CAN), histo-incompatibility, degree of sensitization, donor's age and the like.

In one embodiment, the method comprises a step of providing a sample from the subject.

The term “sample” generally refers to any sample from a subject, which may be tested for expression levels of a biomarker.

In one embodiment, the sample is a body tissue or a bodily fluid sample.

In one embodiment, the sample is a body tissue sample. Body tissue samples taken from a subject are also termed “biopsy”. Examples of body tissues include, but are not limited to, kidney, liver, muscle, heart, lung, pancreas, spleen, thymus, esophagus, stomach, intestine, brain, nerve, testis, prostate, ovary, hair, skin, bone, breast, uterus, bladder and spinal cord.

In one embodiment, the sample is a kidney tissue sample.

In one embodiment, the sample is a bodily fluid. Examples of bodily fluids include, but are not limited to, blood, plasma, serum, lymph, ascetic fluid, cystic fluid, urine, bile, nipple exudate, synovial fluid, bronchoalveolar lavage fluid, sputum, amniotic fluid, peritoneal fluid, cerebrospinal fluid, pleural fluid, pericardial fluid, semen, saliva, sweat, feces, stools, and alveolar macrophages.

In one embodiment, the sample is a bodily fluid selected from the group comprising of consisting of blood, plasma and serum.

In one embodiment, the sample was previously taken from the subject, i.e., the method of the invention does not comprise a step of taking a sample from the subject. Consequently, according to this embodiment, the method of the invention is a non-invasive method or “in vitro method”.

In one embodiment, the method comprises a step of determining the level, amount or concentration of at least one biomarker selected from the group comprising or consisting of TCL1A and AKR1C3 in the sample.

In one embodiment, the method comprises a step of determining the level, amount or concentration of TCL1A in the sample.

In one embodiment, the method comprises a step of determining the level, amount or concentration of AKR1C3 in the sample.

In one embodiment, the method comprises a step of determining the levels, amounts or concentrations of TCL1A and AKR1C3 in the sample.

In one embodiment, the method comprises a step of determining the level, amount or concentration of at most two biomarkers selected from the group comprising or consisting of TCL1A and AKR1C3 in the sample. Hence, in one embodiment, the method does not comprise determining the levels, amounts or concentrations of biomarkers other than TCL1A and/or AKR1C3 in the sample. In particular, the method does not comprise determining the levels, amounts or concentrations of any of CD40, CTLA4, ID3, and MZB1.

In one embodiment, the level, amount or concentration corresponds to the transcription level (i.e., the expression of mRNA) or to the translation level (i.e., the expression of the corresponding protein) of the at least one biomarker.

In one embodiment, the level, amount or concentration of the at least one biomarker is determined at the RNA level, i.e., at the transcription level. Methods for determining the transcription level of a biomarker are well known in the art. Examples of such methods include, but are not limited to, real-time quantitative PCR (qPCR), RT-PCR, RT-qPCR, hybridization techniques (such as, e.g., using microarrays, NanoString® method, etc.), northern blot, and combination thereof, including, but not limited to, hybridization of amplicons obtained by RT-PCR, sequencing (such as, e.g., next-generation DNA sequencing or RNA-seq—also known as “whole transcriptome shotgun sequencing”), and the like.

In one embodiment, the level, amount or concentration of the at least one biomarker is determined at the protein level, i.e., at the translation level. Methods for determining the translation level of a biomarker are well known in the art. Examples of such methods include, but are not limited to, immunohistochemistry, multiplex methods (Luminex), western blot, enzyme-linked immunosorbent assay (ELISA), sandwich ELISA, flow cytometry, fluorescent-linked immunosorbent assay (FLISA), enzyme immunoassay (EIA), radioimmunoassay (RIA), mass spectrometry (such as, e.g., tandem mass spectrometry [MS/MS], chromatography-assisted mass spectrometry and combinations thereof), and the like.

In one embodiment, the level, amount or concentration can be expressed in terms of absolute or relative levels, amounts or concentrations.

When expressed in terms of relative levels, amounts or concentrations, the levels, amounts or concentrations are normalized relative to the level, amount or concentration of one or several reference markers. A “reference marker” may also be referred to as “housekeeping marker” and can be a “housekeeping gene” if the level, amount or concentration of the at least one biomarker is determined at the RNA level; or a “housekeeping protein” if the level, amount or concentration of the at least one biomarker is determined at the protein level. The term “housekeeping marker” hence refers to a gene or a protein, constitutively expressed and necessary for basic maintenance and essential cellular functions. A housekeeping marker is generally not expressed in a cell- or tissue-dependent manner, most often being expressed by all cells in a given organism. Housekeeping markers also have a relatively stable or steady expression; hence they serve as suitable markers to normalize levels, amounts or concentrations of biomarkers of interest. Housekeeping markers and their use in data normalization are well known in the art.

In one embodiment, the method comprises a step of determining a composite score with the level, amount or concentration of the at least one biomarker selected from the group comprising or consisting of TCL1A and AKR1C3, preferably of the two of TCL1A and AKR1C3 biomarkers, as described above.

In one embodiment, the composite score (hereafter “SCR score”) is established using the following formula (1):

$$SCR score = \sum {β}_{i} {X}_{i} + {β}_{0} \qquad ( 1 )$$

wherein:
“βᵢ” represents the regression coefficient for each predictor i among the level, amount or concentration of the biomarkers;
“Xᵢ” represent the predictor variable (also termed independent variable, x-variable or input variable) for each predictor i among the level, amount or concentration of the biomarkers;
“β₀” represents the intercept of the equation, i.e., the value of the criterion when the predictor variables are equal to zero.

In one embodiment, the regression coefficient βᵢ for each predictor i is established using the following formula (4):

$$Regression {coefficient}_{predictor} = log ( odds {ratio}_{predictor} ) \qquad ( 4 )$$

wherein:
“odds ratio_{predictor}” represents the odds ratio for a given predictor.

As used herein, the term “odds ratio” refers to the strength of the association between two events, in particular, between the predictor and the given disease or condition, i.e., subclinical rejection. In other words, odds ratio can be defined as the ratio of the odds of the given disease or condition, i.e., subclinical rejection in the presence of the predictor and the odds of the predictor in the absence of the given disease or condition, i.e., subclinical rejection, or vice versa. If the odds ratio is greater than 1, then the two events are positively correlated. Conversely, if the odds ratio is less than 1, then the two events are negatively correlated.

Odds ratio may be determined, e.g., by univariate or multivariate logistic regression analysis of each predictor with the diagnosis of the given disease or condition, i.e., subclinical rejection, as shown in the Example section.

In one embodiment, the odds ratio may be the ratio of the odds of a subject being affected with subclinical rejection.

The SCR score established using formula (1) is particularly suitable for diagnosing subclinical rejection broadly speaking, i.e., subclinical T-cell mediated kidney rejection (sTCMR), subclinical antibody-mediated kidney rejection (sABMR), and/or mixed sTCMR/sABMR; as demonstrated in Example 1 below.

Additionally or alternatively, the method comprises a step of determining a SCR score, with:
the level, amount or concentration of the at least one biomarker selected from the group comprising or consisting of TCL1A and AKR1C3, preferably of the two of TCL1A and AKR1C3 biomarkers, as described above; and
one, two, three, or four clinical parameters, preferably three or four clinical parameters.

In one embodiment, the clinical parameters are not selected among (i) the age of said kidney recipient subject at test time and (ii) the age of said kidney recipient subject at transplantation time.

In one embodiment, the clinical parameters are selected among:
the experience of rejection episodes before blood sampling (yes/no);
the recipient gender (M/F);
the uptake of immunosuppressant (IS) at blood sampling (yes/no);
the allograft rank, also referred to as experience of previous transplantation (no previous transplantation/1 or more previous transplantations); and
the number of HLA-A, -B and/or -DR mismatches (0-3/>3).

In one embodiment, the clinical parameters are selected among (i) the experience of rejection episodes before blood sampling (yes/no), (ii) the recipient gender (M/F) and (iii) the uptake of immunosuppressant (IS) at blood sampling (yes/no).

In one embodiment, the uptake of immunosuppressant (IS) is the uptake of tacrolimus or the uptake of cyclosporine A (CsA). In one embodiment, the uptake of immunosuppressant (IS) is the uptake of tacrolimus. In one embodiment, the uptake of immunosuppressant (IS) is the uptake of cyclosporine A (CsA).

In one embodiment, the SCR score is established using formula (1) above, wherein:
“βᵢ” represents the regression coefficient for each predictor i among the level, amount or concentration of the biomarkers and the clinical parameters;
“Xᵢ” represent the predictor variable for each predictor i among the level, amount or concentration of the biomarkers and the clinical parameters;
“β₀” represents the intercept of the equation.

In one embodiment, the SCR score is established using the following formula (2):

$$SCR score = \sum ( {β}_{previous rejection episode} \times previous rejection episode + {β}_{IS uptake} \times IS uptake + {β}_{recipient gender} \times recipient gender + {β}_{TCL 1 A} \times Expr ( TCL 1 A ) + {β}_{AKR 1 C 3} \times Expr ( AKR 1 C 3 ) ) + {β}_{0} \qquad ( 2 )$$

wherein:
“β_{TCL1A}”, “β_{AKR1C3}”, “β_{previous rejection episode}”, “β_{IS uptake}”, and “β_{recipient gender}” represent the regression coefficients for each predictor among the level, amount or concentration of the biomarkers and the clinical parameters;
“previous rejection episode” represents the predictor variable defining the experience of rejection episodes before blood sampling, with 0=no previous rejection episodes and 1=at least one or several previous rejection episodes;
“IS uptake” represents the predictor variable defining the uptake of immunosuppressant at blood sampling time, with 0=no CsA uptake or alternatively tacrolimus uptake, and 1=CsA uptake or alternatively no tacrolimus uptake;
“recipient gender” represents the predictor variable defining the gender of the graft recipient, with 0=female and 1=male;
“Expr(TCL1A)” and “Expr(AKR1C3)” represent the predictor variables defining the level, amount or concentration of TCL1A and AKR1C3, respectively;
“β₀” represents the intercept of the equation, i.e., the value of the criterion when the predictor variables are equal to zero.

In one embodiment, the uptake of immunosuppressant (IS) is the uptake of tacrolimus, and formula (2) reads:

$$SCR score = \sum ( {β}_{previous rejection episode} \times previous rejection episode + {β}_{tacrolimus uptake} \times tacrolimus uptake + {β}_{recipient gender} \times recipient gender + {β}_{TCL 1 A} \times Expr ( TCL 1 A ) + {β}_{AKR 1 C 3} \times Expr ( AKR 1 C 3 ) ) + {β}_{0} \qquad ( 2 )$$

wherein:
“β_{tacrolimus uptake}” represents the regression coefficients for the predictor “uptake of tacrolimus at blood sampling time”; and
“tacrolimus uptake” represents the predictor variable defining the uptake of tacrolimus at blood sampling time, with 0=tacrolimus uptake, and 1=no tacrolimus uptake.

In one embodiment, the uptake of immunosuppressant (IS) is the uptake of cyclosporine A (CsA), and formula (2) reads:

$$SCR score = \sum ( {β}_{previous rejection episode} \times previous rejection episode + {β}_{CsA uptake} \times C s A uptake + {β}_{recipient gender} \times recipient gender + {β}_{TCL 1 A} \times Expr ( TCL 1 A ) + {β}_{AKR 1 C 3} \times Expr ( AKR 1 C 3 ) ) + {β}_{0} \qquad ( 2 )$$

wherein:
“β_{CsA uptake}” represents the regression coefficients for the predictor “uptake of CsA at blood sampling time”; and
“CsA uptake” represents the predictor variable defining the uptake of cyclosporin A at blood sampling time, with 0=no CsA uptake, and 1=CsA uptake.

(3) Regression coefficient_{predictor}=log(odds ratio_{predictor}) In one embodiment, wherein the odds ratio are the ratio of the odds of a subject being affected with a subclinical rejection, odds ratio_{TCL1A}, odds ratio_{AKR1C3}, odds ratio_{recipient gender} and odds ratio_{tacrolimus} uptake are less than 1.

In one embodiment, wherein the odds ratio are the ratio of the odds of a subject being affected with a subclinical rejection, odds ratio_{previous rejection episode} and odds ratio_{CsA uptake} are greater than 1.

In an exemplary embodiment, odds ratios of a subject being affected with subclinical rejection are as defined in Table 3. In an exemplary, odds ratios of a subject being affected with subclinical rejection are within the 95% confidence level as defined in Table 3.

Alternatively, the odds ratio may be the ratio of the odds of a subject not being affected with subclinical rejection. According to this embodiment, it is expectable that odd ratios defined above for a subject being affected with subclinical rejection be reversed, i.e., that odds ratio_{TCL1A}, odds ratio_{AKR1C3}, odds ratio_{recipient gender} and odds ratio_{tacrolimus uptake} be greater than 1, and that odds ratio_{previous rejection episode} and odds ratio_{CsA uptake} be less than 1.

The SCR score established using formula (2) is particularly suitable for diagnosing subclinical rejection broadly speaking, i.e., subclinical T-cell mediated kidney rejection (sTCMR), subclinical antibody-mediated kidney rejection (sABMR), and/or mixed sTCMR/sABMR; as demonstrated in Example 1 below.

In one embodiment, the SCR score is established using the following formula (3):

$$SCR score = \sum ( {β}_{previous rejection episode} \times previous rejection episode + {β}_{allograft rank} \times allograft rank + {β}_{HLA mismatches} \times HLA mismatches + {β}_{recipient gender} \times recipient gender + {β}_{TCL 1 A} \times Expr ( TCL 1 A ) + {β}_{AKR 1 C 3} \times Expr ( AKR 1 C 3 ) ) + {β}_{0} \qquad ( 3 )$$

wherein:
“β_{TCL1A}”, “β_{AKR1C3}”, “β_{previous rejection episode}”, “β_{allograft rank}”, “β_{HLA mismatches}”, and “β_{recipient gender}” represent the regression coefficients for each predictor among the level, amount or concentration of the biomarkers and the clinical parameters;
“previous rejection episode” represents the predictor variable defining the experience of rejection episodes before blood sampling, with 0=“no previous rejection episodes” and 1=“one or several previous rejection episodes”;
“allograft rank” represents the predictor variable defining the occurrence of previous transplantation, with 0=“no previous transplantation” and 1=“one or several previous transplantations”;
“HLA mismatches” represents the predictor variable defining the occurrence of donor-recipient HLA mismatches, with 0=“3 or less HLA-A, -B and/or -DR mismatches”, and 1=“more than 3 HLA-A, -B and/or -DR mismatches”;
“recipient gender” represents the predictor variable defining the gender of the graft recipient, with 0=“female” and 1=“male”;
“Expr(TCL1A)” and “Expr(AKR1C3)” represent the predictor variables defining the level, amount or concentration of TCL1A and AKR1C3, respectively;
“β₀” represents the intercept of the equation;

In one embodiment, wherein the odds ratio are the ratio of the odds of a subject being affected with a subclinical rejection, odds ratio_{TCL1A}, odds ratio_{AKR1C3} and odds ratio_{recipient gender} are less than 1.

In one embodiment, wherein the odds ratio are the ratio of the odds of a subject being affected with a subclinical rejection, odds ratio_{previous rejection episode}, odds ratio_{allograft rank} and odds ratio_{HLA mismatches} are greater than 1.

Alternatively, the odds ratio may be the ratio of the odds of a subject not being affected with subclinical rejection. According to this embodiment, it is expectable that odd ratios defined above for a subject being affected with subclinical rejection be reversed, i.e., that odds ratio_{TCL1A}, odds ratio_{AKR1C3} and odds ratio_{recipient gender} be greater than 1, and that odds ratio_{previous rejection episode}, odds ratio_{allograft rank} and odds ratio_{HLA mismatches} be less than 1.

In an exemplary embodiment, odds ratios of a subject not being affected with subclinical rejection are as defined in FIG. 15A.

The SCR score established using formula (3) is particularly suitable for diagnosing a specific subtype of subclinical rejection: subclinical antibody-mediated kidney rejection (sABMR), as demonstrated in Example 2 below.

In one embodiment, the method comprises a step of comparing the level, amount or concentration of the at least one biomarker with the level, amount or concentration of the same at least one biomarker determined in at least one reference subject.

In one embodiment, the reference subject is the subject themselves, prior to kidney transplantation.

In one embodiment, the reference subject is a substantially healthy subject, preferably a subject who has not undergone kidney transplantation.

In one embodiment, the reference subject is a kidney transplant recipient not affected with subclinical rejection.

By implying a multitude of samples from several reference subjects, it is also conceivable to calculate a median and/or mean level, amount or concentration of the at least one biomarker.

Hence, in one embodiment, the method comprises a step of comparing the level, amount or concentration of the at least one biomarker with the median and/or mean level, amount or concentration of the same at least one biomarker determined in a reference population.

In one embodiment, the reference population comprises or consists of two or more, such as, 2, 5, 10, 20, 30, 40, 50 or more, substantially healthy subjects, preferably two or more subjects who have not undergone kidney transplantation.

In one embodiment, the reference population comprises or consists of two or more, such as, 2, 5, 10, 20, 30, 40, 50 or more, kidney transplant subjects not affected with subclinical rejection.

In one embodiment, the method comprises a step of comparing the level, amount or concentration of the at least one biomarker with:
the level, amount or concentration of the same at least one biomarker determined in at least one reference subject as defined above, or the median and/or mean level, amount or concentration of the same at least one biomarker determined in a reference population as defined above; and
the level, amount or concentration of the same at least one biomarker determined in at least one subject known to be affected with subclinical rejection, or the median and/or mean level, amount or concentration of the same at least one biomarker determined in a population of subjects known to be affected with subclinical rejection.

Additionally or alternatively, the method comprises a step of comparing the composite score with a reference composite score determined in at least one reference subject.

In one embodiment, the reference subject is the subject themselves, prior to kidney transplantation.

In one embodiment, the reference subject is a substantially healthy subject, preferably a subject who has not undergone kidney transplantation.

In one embodiment, the reference subject is a kidney transplant recipient not affected with subclinical rejection.

By implying a multitude of samples from several reference subjects, it is also conceivable to calculate a median and/or mean reference composite score.

Hence, in one embodiment, the method comprises a step of comparing the composite score with the median and/or mean reference composite score determined in a reference population.

In one embodiment, the reference population comprises or consists of two or more, such as, 2, 5, 10, 20, 30, 40, 50 or more, substantially healthy subjects, preferably two or more subjects who have not undergone kidney transplantation.

In one embodiment, the reference population comprises or consists of two or more, such as, 2, 5, 10, 20, 30, 40, 50 or more, kidney transplant subjects not affected with subclinical rejection.

In one embodiment, the method comprises a step of comparing the composite score with:
the reference composite score determined in at least one reference subject as defined above, or the median and/or mean reference composite score determined in a reference population as defined above; and
the composite score determined in at least one subject known to be affected with subclinical rejection, or the median and/or mean composite score determined in a population of subjects known to be affected with subclinical rejection.

In one embodiment, the method comprises a step of concluding that the subject is affected with subclinical rejection based on the comparison in the previous step. Alternatively, the method may comprise a step of concluding that the subject is not affected with subclinical rejection based on the comparison in the previous step.

In one embodiment, subclinical rejection is subclinical T-cell mediated rejection (sTCMR) or subclinical antibody-mediated rejection (sABMR). In one embodiment, subclinical rejection is subclinical T-cell mediated rejection (sTCMR). In one embodiment, subclinical rejection is subclinical antibody-mediated rejection (sABMR). In one embodiment, subclinical rejection is mixed sTCMR/sABMR.

In one embodiment, it is concluded that the subject is affected with subclinical rejection when the level, amount or concentration of the at least one biomarker among TCL1A and AKR1C3 is substantially lower than the level, amount or concentration of the same at least one biomarker determined in at least one reference subject as defined above, or than the median and/or mean level, amount or concentration of the same at least one biomarker determined in a reference population as defined above.

In one embodiment, it is concluded that the subject is affected with subclinical rejection when the level, amount or concentration of TCL1A is substantially lower than the level, amount or concentration of TCL1A determined in at least one reference subject as defined above, or than the median and/or mean level, amount or concentration of TCL1A determined in a reference population as defined above.

In one embodiment, it is concluded that the subject is affected with subclinical rejection when the level, amount or concentration of AKR1C3 is substantially lower than the level, amount or concentration of AKR1C3 determined in at least one reference subject as defined above, or than the median and/or mean level, amount or concentration of AKR1C3 determined in a reference population as defined above.

In one embodiment, it is concluded that the subject is affected with subclinical rejection when the level, amount or concentration of both TCL1A and AKR1C3 biomarkers is substantially lower than the level, amount or concentration of both TCL1A and AKR1C3 determined in at least one reference subject as defined above, or than the median and/or mean level, amount or concentration of both TCL1A and AKR1C3 determined in a reference population as defined above.

By “substantially lower”, it is meant that the absolute or relative level, amount or concentration of a given biomarker is statistically significantly decreased compared to the same biomarker in the at least one reference subject or in the reference population, such as, e.g., decreased by 10%, 20%, 30%, 40%, 50%, 60%, 70%, 80% or more compared to the same biomarker in the at least one reference subject or in the reference population.

In one embodiment, it is concluded that the subject is not affected with subclinical rejection when the level, amount or concentration of the at least one biomarker among TCL1A and AKR1C3 is substantially equal to or higher than the level, amount or concentration of the same at least one biomarker determined in at least one subject known to be affected with subclinical rejection, or the median and/or mean level, amount or concentration of the same at least one biomarker determined in a population of subjects known to be affected with subclinical rejection.

In one embodiment, it is concluded that the subject is not affected with subclinical rejection when the level, amount or concentration of TCL1A is substantially equal to or higher than the level, amount or concentration of TCL1A determined in at least one subject known to be affected with subclinical rejection, or the median and/or mean level, amount or concentration of TCL1A determined in a population of subjects known to be affected with subclinical rejection.

In one embodiment, it is concluded that the subject is not affected with subclinical rejection when the level, amount or concentration of AKR1C3 is substantially equal to or higher than the level, amount or concentration of AKR1C3 determined in at least one subject known to be affected with subclinical rejection, or the median and/or mean level, amount or concentration of AKR1C3 determined in a population of subjects known to be affected with subclinical rejection.

In one embodiment, it is concluded that the subject is not affected with subclinical rejection when the level, amount or concentration of both TCL1A and AKR1C3 biomarkers is substantially equal to or higher than the level, amount or concentration of both TCL1A and AKR1C3 determined in at least one subject known to be affected with subclinical rejection, or the median and/or mean level, amount or concentration of both TCL1A and AKR1C3 determined in a population of subjects known to be affected with subclinical rejection.

By “substantially equal”, it is meant that the absolute or relative level, amount or concentration of a given biomarker is not statistically significantly different than the level, amount or concentration of the same biomarker in the at least one subject known to be affected with subclinical rejection or in the population of subjects known to be affected with subclinical rejection, such as, e.g., is within ±10% of the level, amount or concentration of the same biomarker in the at least one subject known to be affected with subclinical rejection or in the population of subjects known to be affected with subclinical rejection.

By “substantially higher”, it is meant that the absolute or relative level, amount or concentration of a given biomarker is statistically significantly increased compared to the same biomarker in the at least one subject known to be affected with subclinical rejection or in the population of subjects known to be affected with subclinical rejection, such as, e.g., by 10%, 20%, 30%, 40%, 50%, 60%, 70%, 80% or more compared to the same biomarker in the at least one subject known to be affected with subclinical rejection or in the population of subjects known to be affected with subclinical rejection.

Additionally or alternatively, it is concluded that the subject is affected with subclinical rejection when the composite score is substantially higher than the reference composite score determined in at least one reference subject as defined above, or than the median and/or mean reference composite score determined in a reference population as defined above.

By “substantially higher”, it is meant that the composite score is statistically significantly increased compared to the reference composite score of the at least one reference subject or reference population, such as, e.g., by 10%, 20%, 30%, 40%, 50%, 60%, 70%, 80% or more compared to the reference composite score of the at least one reference subject or reference population.

In one embodiment, it is concluded that the subject is not affected with subclinical rejection when the composite score is substantially equal to or lower than the composite score determined in at least one subject known to be affected with subclinical rejection, or the median and/or mean composite score determined in a population of subjects known to be affected with subclinical rejection.

By “substantially equal”, it is meant that the composite score is not statistically significantly than the composite score in the at least one subject known to be affected with subclinical rejection or in the population of subjects known to be affected with subclinical rejection, such as, e.g., is within +10% of the composite score in the at least one subject known to be affected with subclinical rejection or in the population of subjects known to be affected with subclinical rejection.

By “substantially lower”, it is meant that the composite score is statistically significantly decreased compared to the composite score of the at least one subject known to be affected with subclinical rejection or of the population of subjects known to be affected with subclinical rejection, such as, e.g., by 10%, 20%, 30%, 40%, 50% or more compared to the composite score of the at least one subject known to be affected with subclinical rejection or of the population of subjects known to be affected with subclinical rejection.

The present invention also relates to a method of treating subclinical rejection in a subject in need thereof.

The term “treatment” and its declensions refer to the administration to a subject of a therapeutic regimen in order to abrogate, inhibit, slow or reverse the progression of a given disease or condition, and/or to ameliorate clinical symptoms of the given disease or condition, and/or to prevent the appearance of further clinical symptoms of the given disease or condition, e.g., subclinical rejection. In particular, subclinical rejection may be detrimental to a graft and, if left untreated, may progress to chronic allograft nephropathy (CAN), chronic interstitial fibrosis and tubular atrophy, renal dysfunction, reduced creatinine clearance, chronic rejection, and ultimately, shorter graft survival.

In one embodiment, the method comprises a first step of diagnosing subclinical rejection in the subject, using the method detailed above.

In one embodiment, the method comprises a second step of treating the subject with an immunosuppressive therapy if or when said subject is diagnosed with subclinical rejection during the first step.

In one embodiment, treating the subject diagnosed with subclinical rejection comprises resuming a previously-completed immunosuppressive therapy.

In one embodiment, treating the subject diagnosed with subclinical rejection comprises increasing the dosage regimen of a currently-administered immunosuppressive therapy.

In one embodiment, treating the subject diagnosed with subclinical rejection comprises changing the currently-administered immunosuppressive therapy for a more aggressive treatment.

In one embodiment, treating the subject diagnosed with subclinical rejection comprises administering another immunosuppressive therapy on top of the currently-administered immunosuppressive therapy.

Examples of suitable immunosuppressive therapies are fully detailed earlier in the specification.

As an exemplary therapeutic protocol of immunosuppressive therapy, kidney transplant recipients typically receive an induction treatment consisting of 2 injections of basiliximab, in association with tacrolimus (0.1 mg/kg/day), mycophenolate mofetil (2 g/day) and corticoids (1 mg/kg/day), the corticoid treatment being progressively decreased by 10 mg every 5 days until the end of treatment. A more aggressive protocol would include a short course of anti-thymocyte globulin (e.g., from day 0 to day 7) followed by tacrolimus (0.1 mg/kg/day; e.g., from day 7 to the end of the treatment), in association from day 0 with mycophenolate mofetil (2 g/day) and corticosteroids (1 mg/kg/day), the corticoid treatment being progressively decreased by 10 mg every 5 days until the end of treatment.

In one embodiment, treating the subject diagnosed with subclinical rejection comprises removing or reducing the number of immunoglobulins in the subject, e.g., by plasma exchange (PLEX) or by administration of an IgG-degrading enzyme (such as imlifidase); and optionally further administering IVIg (intravenous immune globulins). This course of treatment may be particularly suitable when the subject is diagnosed with antibody-mediated rejection (sABMR).

In one embodiment, treating the subject diagnosed with subclinical rejection comprises administering anti-thymocyte globulin (ATG) ad/or T-cell-depleting antibodies. This course of treatment may be particularly suitable when the subject is diagnosed with antibody-mediated rejection (sABMR).

In one embodiment, treating the subject diagnosed with subclinical rejection comprises performing surgical splenectomy, splenic embolization and/or splenic radiation of the subject's spleen.

In one embodiment, treating the subject diagnosed with subclinical rejection comprises administering complement inhibitors. Some examples of complement inhibitors include, but are not limited to, C5 inhibitors (such as the anti-C5 antibody eculizumab), or C1 esterase inhibitor. This course of treatment may be particularly suitable when the subject is diagnosed with antibody-mediated rejection (sABMR).

For treatments specific to sABMR, reference is also made to Schinstock et al., 2020 (Transplantation. 104(5):911-922), the content of which is incorporated by reference.

The skilled artisan will readily appreciate that these courses of treatment are not exclusive and can be combined, within a physician's scope of sound medical judgment.

The present invention also relates to a method for identifying a subject under immunosuppressive therapy as a candidate for immunosuppressive therapy weaning or minimization.

In one embodiment, the method comprises a first step of diagnosing subclinical rejection in the subject, using the method detailed above.

In one embodiment, the method comprises a second step of reducing and eventually suppressing an immunosuppressive therapy in the subject if or when said subject is not diagnosed with subclinical rejection during the first step, and preferably further if or when said subject was determined to be an operationally tolerant kidney transplant recipient. Means and methods of determining whether a kidney transplant recipient is operationally tolerant or not have been described in the art, in particular in WO 2018/015551 or in Danger et al., 2017 (Kidney Int. 91(6): 1473-1481).

The present invention also relates to a computer system for diagnosing subclinical rejection in a subject in need thereof. The present invention also related to a computer-implemented method for diagnosing subclinical rejection in a subject in need thereof.

As used herein, the term “computer system” refers to any and all devices capable of storing and processing information and/or capable of using the stored information to control the behavior or execution of the device itself, regardless of whether such devices are electronic, mechanical, logical, or virtual in nature. The term “computer system” can refer to a single computer, but also to a plurality of computers working together to perform the function described as being performed on or by a computer system. A method implemented using a computer system is referred to as a “computer-implemented method”.

In one embodiment, the computer system according to the invention comprises:
(ii) at least one processor, and
(iii) at least one computer-readable storage medium that stores code readable by the processor.

As used herein, the term “processor” is meant to include any integrated circuit or other electronic device capable of performing an operation on at least one instruction word, such as, e.g., executing instructions, codes, computer programs, and scripts which it accesses from a storage medium. However, the term “processor” should not be construed to be restricted to hardware capable of executing software, and refers in a general way to a processing device, which can for example include a computer, a microprocessor, an integrated circuit, or a programmable logic device (PLD). The processor may also encompass one or more graphics processing units (GPU), whether exploited for computer graphics and image processing or other functions. Additionally, the instructions and/or data enabling to perform associated and/or resulting functionalities may be stored on any processor-readable medium, including, but not limited to, an integrated circuit, a hard disk, a magnetic tape (including floppy disk and zip diskette), an optical disc (including Blu-ray, compact disc and digital versatile disc), a flash memory (including memory card and USB flash drive) a random-access memory (RAM) (including dynamic and static RAM), a read-only memory (ROM) or a cache. Instructions may be in particular stored in hardware, software, firmware or in any combination thereof.

Examples of processors include, but are not limited to, central processing units (CPU), microprocessors, digital signal processors (DSPs), general purpose microprocessors, application specific integrated circuits (ASICs), field programmable logic arrays (FPGAs), and other equivalent integrated or discrete logic circuitry.

In one embodiment, the computer system according to the invention is linked to a scanner or the like receiving experimentally determined signals related to the level, amount or concentration of the at least one biomarker selected from the group comprising or consisting of TCL1A and AKR1C3.

Alternatively, level, amount or concentration of the at least one biomarker selected from the group comprising or consisting of TCL1A and AKR1C3 in the sample expression levels can be input by other means, optionally along with the one, two, or preferably three clinical parameters defined above.

The present invention also relates to a computer program comprising software code readable by the processor adapted to perform, when executed by said processor, the computer-implemented method as described herein.

In one embodiment, the computer system according to the invention includes at least one computer program. A computer program may include a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. A computer program may be written in various versions of various languages.

In one embodiment, a computer program includes, in part or in whole, one or more web applications, one or more mobile applications, one or more standalone applications, one or more web browser plug-ins, extensions, add-ins, or add-ons, or combinations thereof.

The present invention also relates to a computer-readable storage medium comprising code readable by the processor which, when executed by said processor, causes the processor to carry out the steps of the computer-implemented methods as described herein.

Examples of computer-readable storage medium include, but are not limited to, an integrated circuit, a hard disk, a magnetic tape (including floppy disk and zip diskette), an optical disc (including Blu-ray, compact disc and digital versatile disc), a flash memory (including memory card and USB flash drive) a random-access memory (RAM) (including dynamic and static RAM), a read-only memory (ROM) or a cache.

In one embodiment, the computer-readable storage medium is a non-transitory computer-readable storage medium.

In one embodiment, the code stored on the computer-readable storage medium, when executed by the processor of the computer system, causes the processor to:
a. receive an input level, amount or concentration of the at least one biomarker selected from the group comprising or consisting of TCL1A and AKR1C3,
b. analyze and transform the input level, amount or concentration by organizing and/or modifying each input level to derive at least one of a probability score, a fitting score and a classification label,
c. generate an output, wherein the output is the at least one of the classification label, the fitting score and the probability score, and
d. provide a diagnosis of the subject as being affected or not with subclinical rejection based on the output.

In one embodiment, the at least one of the classification label, the fitting score and the probability score is the composite score “SCR score” with formula (1) as defined above.

In one embodiment, the code stored on the computer-readable storage medium, when executed by the processor of the computer system, causes the processor to:
a. receive input levels, amounts or concentrations of the at least one biomarker selected from the group comprising or consisting of TCL1A and AKR1C3, and input values for one, two, three, or four clinical parameters, preferably of three or four clinical parameters, as defined above,
b. analyze and transform the input levels, amounts or concentrations, and the input values, by organizing and/or modifying each input to derive at least one of a probability score, a fitting score and a classification label,
c. generate an output, wherein the output is the at least one of the classification label, the fitting score and the probability score, and
d. provide a diagnosis of the subject as being affected or not with subclinical rejection based on the output.

In one embodiment, the at least one of the classification label, the fitting score and the probability score is the composite score “SCR score” with formula (2) as defined above.

In one embodiment, the at least one of the classification label, the fitting score and the probability score is the composite score “SCR score” with formula (3) as defined above.

The present invention also relates to a kit-of-parts.

As used herein, the term “kit-of-parts” refers to an article of manufacture comprising one or more containers filled with one or more means or reagents for performing the methods according to the invention.

In one embodiment, the kit-of-parts comprises of at least one means for determining the level, amount or concentration of at least one biomarker selected from the group comprising or consisting of TCL1A and AKR1C3 in a sample. Such means may be, e.g., probes for determining the level, amount or concentration of the at least one biomarker at the RNA or protein level.

In one embodiment, the kit-of-parts comprises at least one means for determining the level, amount or concentration of at least one reference marker as described above. Such means may be, e.g., probes for determining the level, amount or concentration of the at least one reference marker at the RNA or protein level.

In one embodiment, the kit-of-parts does not comprise means for determining the level, amount or concentration of any other biomarker than TCL1A and AKR1C3, and optionally of the at least one reference marker. In particular, the kit-of-parts does not comprise means for determining the level, amount or concentration of any of CD40, CTLA4, ID3, and MZB1.

Examples of probes for determining the level, amount or concentration of the at least one biomarker and/or of the at least one reference marker at the RNA level include, but are not limited to, nucleic acid probes (such as, e.g., TaqMan™ probes, NanoString probes, Scorpions® probes, Molecular Beacons, and LNAR (Locked Nucleic Acid) probes).

Examples of probes for determining the level, amount or concentration of the at least one biomarker and/or of the at least one reference marker at the protein level include, but are not limited to, antibodies (such as, e.g., anti-AKR1C3 antibodies and anti-TCL1A antibodies) and aptamers.

In one embodiment, the probes may be immobilized on a solid support, such as, e.g., an array.

In one embodiment, the probes comprise at least one detectable label. Examples of suitable detectable labels include, but are not limited to, FAM (5- or 6-carboxyfluorescein), HEX, CY5, VIC, NED, fluorescein, FITC, IRD-700/800, CY3, CY3.5, CY5.5, TET (5-tetrachloro-fluorescein), TAMRA, JOE, ROX, BODIPY TMR, Oregon Green, Rhodamine Green, Rhodamine Red, Texas Red, Yakima Yellow, Alexa Fluor PET, BIOSEARCH BLUE™, MARINA BLUER, BOTHELL BLUER, ALEXA FLUORR, 350 FAM™, SYBRR Green 1, EvaGreen™, ALEXA FLUORR 488 JOE™, 25 VIC™, HEX™, TET™, CAL FLUOR® Gold 540, YAKIMA YELLOWR, ROX™, CAL FLUOR® Red 610, Cy3.5™, TEXAS REDR, ALEXA FLUORR 568 CRY5™, QUASAR™ 670, LIGHTCYCLER RED640R, ALEXA FLUORR 633 QUASAR™ 705, LIGHTCYCLER RED705R, ALEXA FLUORR 680, SYTOR9, LC GREENR, LC GREEN® Plus+, and EVAGREEN™.

Also disclosed herein are:
E1: A method of diagnosing subclinical rejection in a subject in need thereof, comprising the steps of:
a) determining the level, amount or concentration of at least one biomarker selected from the group consisting of AKR1C3 and TCL1A in a sample previously taken from the subject;
b) comparing the level, amount or concentration of the at least one biomarker with the level, amount or concentration of the same at least one biomarker determined in at least one reference subject; and
c) concluding that the subject is affected with subclinical rejection when the level, amount or concentration of the at least one biomarker is statistically significantly lower than the level, amount or concentration of the same at least one biomarker determined in at least one reference subject.
E2: The method according to E1, wherein step a) comprises determining the level, amount or concentration of AKR1C3 in the sample previously taken from the subject.
E3: The method according to E1, wherein step a) comprises determining the level, amount or concentration of TCL1A in the sample previously taken from the subject.
E4: The method according to any one of E1 to E3, wherein step a) comprises determining the level, amount or concentration of both AKR1C3 and TCL1A in the sample previously taken from the subject.
E5: The method according to any one of E1 to E4, wherein the level, amount or concentration of the at least one biomarker is expressed in terms of absolute or relative levels, amounts or concentrations; preferably is expressed in terms of relative levels, amounts or concentrations normalized relative to the level, amount or concentration of one or several reference markers.
E6: The method according to any one of E1 to E5, comprising:
a) determining a composite score, with:
   the level, amount or concentration of the at least one biomarker selected from the group consisting of AKR1C3 and TCL1A, preferably of both AKR1C3 and TCL1A; and
   one, two, or preferably three clinical parameters;
b) comparing the composite score with a reference composite score determined in at least one reference subject;
c) concluding that the subject is affected with subclinical rejection when the composite score is substantially higher than the reference composite score determined in at least one reference subject.
E7: The method according to E6, wherein the clinical parameters are selected among (i) the experience of rejection episodes before blood sampling, (ii) the recipient gender and (iii) the uptake of cyclosporine A (CsA) at blood sampling.
E8: The method according to E6 or E7, wherein the composite score is established using the following formula:

$$Score = \sum {β}_{previous rejection episode} \times previous rejection episode + {β}_{CsA uptake} \times C s A uptake + {β}_{recipient gender} \times recipient gender + {β}_{TCL 1 A} \times Expr ( TCL 1 A ) + {β}_{AKR 1 C 3} \times Expr ( AKR 1 C 3 ) + {β}_{0}$$

wherein:
   “β_{TCL1A}”, “β_{AKR1C3}”, “B_{previous rejection episode}”, “β_{CsA uptake}”, and “β_{recipient gender}” represent the regression coefficients for each predictor among the level, amount or concentration of the biomarkers and the clinical parameters;
   “previous rejection episode” represents the predictor variable defining the experience of rejection episodes before blood sampling, with 0=no previous rejection episodes and 1=at least one or several previous rejection episodes;
   “CsA uptake” represents the predictor variable defining the uptake of Cyclosporin A (CsA) at blood sampling time, with 0=no CsA uptake and 1=CsA uptake;
   “recipient gender” represents the predictor variable defining the gender of the graft recipient, with 0=female and 1=male;
   “Expr(TCL1A)” and “Expr(AKR1C3)” represent the predictor variables defining the level, amount or concentration of TCL1A and AKR1C3, respectively;
   “β₀” represents the intercept of the equation.
E9: The method according to any one of E1 to E8, wherein the at least one reference subject is a subject who has not undergone kidney transplantation and/or a kidney transplant recipient not affected with subclinical rejection.
E10: The method according to any one of E1 to E8, wherein the at least one reference subject is the subject themselves, prior to kidney transplantation.
E11: The method according to any one of E1 to E10, wherein the at least one reference subject is a reference population comprising two or more reference subjects.
E12: Disclosed herein is a computer system for diagnosing subclinical rejection in a subject in need thereof, the computer system comprising:
i) at least one processor, and
ii) at least one storage medium that stores at least one code readable by the processor, and which, when executed by the processor, causes the processor to:
   a. receive an input level, amount or concentration of the at least one biomarker selected from the group consisting of AKR1C3 and TCL1A,
   b. analyze and transform the input level, amount or concentration by organizing and/or modifying each input level to derive at least one of a probability score, a fitting score and a classification label,
   c. generate an output, wherein the output is the at least one of the classification label, the fitting score and the probability score, and
   d. provide a diagnosis of the subject as being affected or not with subclinical rejection based on the output.
E13: The computer system according to E12, wherein the at least one code readable by the processor, when executed by the processor, causes the processor to:
a. receive input levels, amounts or concentrations of the at least one biomarker selected from the group consisting of AKR1C3 and TCL1A, and input values for one, two, or preferably three clinical parameters selected among (i) the experience of rejection episodes before blood sampling, (ii) the recipient gender and (iii) the uptake of cyclosporine A (CsA) at blood sampling,
b. analyze and transform the input levels, amounts or concentrations, and the input values, by organizing and/or modifying each input to derive at least one of a probability score, a fitting score and a classification label,
c. generate an output, wherein the output is the at least one of the classification label, the fitting score and the probability score, and
d. provide a diagnosis of the subject as being affected or not with subclinical rejection based on the output.
E14: The computer system according to E13, wherein the at least one of the classification label, the fitting score and the probability score is the composite score as defined in claim 8.
E15: A kit-of-parts for performing the method according to any one of E1 to E11, comprising means for determining the level, amount or concentration of at least one biomarker selected from the group consisting of AKR1C3 and TCL1A, and optionally means for determining the level, amount or concentration of at least one reference marker.

### BRIEF DESCRIPTION OF THE DRAWINGS

FIG. 1 is a flow diagram showing the inclusion criteria of patients for this study.

FIG. 2 is a histology diagnosis representation of the 450 evaluated biopsies from patients with stable function with qPCR gene expression. Histological features of renal biopsies according the 2015 Banff classification (Loupy et al., 2017. Am J Transplant. 17(1):28-41), the 6 histologic classifications (normal, iIFTA, borderline, others, humoral- and cellular-mediated rejections) and the 2 groups (NR and SCR) are colorized in the upper panel. In the lower panel, the scaled −ΔΔCt values from qPCR measures of the 6 genes composing the cSoT are represented with yellow and blue for high and low gene expression.

FIGS. 3A-B are a set of two graphs showing that the one-year cSoT score values are associated with renal function (MDRD). FIG. 3A: from the 450 patients, the cSoT score is significantly associated with renal function (MDRD formula, in mL/min/1.73 m²) at 12-, 24-, 36- and 48-month post-transplantation, as displayed by the r Pearson correlation. P-values and number of analyzed paired are displayed above and within the bars, respectively. FIG. 3B: the dot plot represents 12-month post-transplantation function (MDRD), as a function of the cSoT score values.

FIGS. 4A-B are a set of two violin plots representing the cSoT score in NR and SCR groups (FIG. 4A) and in each of the 6 histology groups (FIG. 4B). p-values from Student's/tests corrected for multitesting comparing NR to SCR (FIG. 4A) and from Kruskal-Wallis with Dunn's post-hoc tests comparing normal versus other groups are shown.

FIGS. 5A-B are a set of two violin plots representing the AKR1C3 expression in NR and SCR groups (FIG. 5A) and in each of the 6 histology groups (FIG. 5B). Gene expression represent the −ΔΔCt values from qPCR measures. p-values from Student's/tests corrected for multitesting comparing NR to SCR (FIG. 5A) and from Kruskal-Wallis with Dunn's post-hoc tests comparing normal versus other groups are shown.

FIGS. 6A-B are a set of two violin plots representing the TCL1A expression in NR and SCR groups (FIG. 6A) and in each of the 6 histology groups (FIG. 6B). Gene expression represent the −ΔΔCt values from qPCR measures. p-values from Student's/tests corrected for multitesting comparing NR to SCR (FIG. 6A) and from Kruskal-Wallis with Dunn's post-hoc tests comparing normal versus other groups are shown.

FIGS. 7A-B are a set of two violin plots representing the 12-month post-transplantation function (MDRD formula, in mL/min/1.73 m²) in NR and SCR groups (FIG. 7A) and in each of the 6 histology groups (FIG. 7B). p-values from Student's/tests corrected for multitesting comparing NR to SCR (FIG. 7A) and from Kruskal-Wallis with Dunn's post-hoc tests comparing normal versus other groups are shown.

FIGS. 8A-D are a set of four violin plots representing the cSoT score (FIG. 8A), AKR1C3 expression (FIG. 8B), TCL1A expression (FIG. 8C) and 12-month post-transplantation function (MDRD formula, in mL/min/1.73 m²) (FIG. 8D) in each of the 6 histology groups among the 150 patients with for cause biopsy and/or serum creatine levels above 160 μmol/L at one-year. Gene expression represents the −ΔΔCt values from qPCR measures. p-values from Kruskal-Wallis with Dunn's post-hoc tests comparing normal versus other histology groups are shown.

FIG. 9 is a forest plot summarizing the logistic regression model for SRC risk. Values indicates odds ratio and * and *** represents p-values of <0.05 and <0.001, respectively.

FIGS. 10A-B are a set of two graphs showing a violin plot displaying composite score (SCR-s) values of NR patients versus SCR patients with a t test p value (FIG. 10A) and ROC curves exhibiting specificity and sensitivity for the SCR-s (thick black curve), the 3 clinical parameters (logistic regression) (hatched curve) and the 12-month post-transplantation function (grey curve) (FIG. 10B). p-values from ROC curve comparisons using bootstrap test with the same number of controls and cases than the original sample are shown.

FIGS. 11A-C are a set of three graphs showing that the composite score SCR-s is capable of discriminating sABMR and sTCMR patients from NR patients. The violin plot displays SCR-s values of NR versus sABMR and sTCMR patients with Kruskal-Wallis with Dunn's post-hoc tests comparing normal to sABMR and sTCMR (FIG. 11A). Corresponding ROC curves comparing normal to sABMR (FIG. 11B) and normal to sTCMR (FIG. 11C) are displayed with AUCs.

FIGS. 12A-B are a set of two violin plots representing the AKR1C3 expression in NR and SCR groups (FIG. 12A) and in each of the 6 histology groups (FIG. 12B). Gene expression represent the −ΔΔCt values for qPCR measures and log 2 of normalized counts for NanoString measures. p-values from Student's t tests corrected for multitesting comparing NR to SCR (FIG. 12A) and from Kruskal-Wallis with Dunn's post-hoc tests comparing normal versus other groups are shown.

FIGS. 13A-B are a set of two violin plots representing the TCL1A expression in NR and SCR groups (FIG. 13A) and in each of the 6 histology groups (FIG. 13B). Gene expression represent the −ΔΔCt values for qPCR measures and log 2 of normalized counts for NanoString measures. p-values from Student's t tests corrected for multitesting comparing NR to SCR (FIG. 13A) and from Kruskal-Wallis with Dunn's post-hoc tests comparing normal versus other groups are shown.

FIGS. 14A-D are a set of four violin plots showing that the cSoT (FIG. 14A), AKR1C3 (FIG. 14B) and TCL1A (FIG. 14C) expression levels are significantly decreased in blood from sAMR patients compared to others, while renal function (MDRD formula, in mL/min/1.73 m²) does not significantly differ between the 2 groups (FIG. 14D). Gene expression represents the −ΔΔCt values from qPCR measures. p values from Mann-Whitney's tests comparing the 2 groups are shown.

FIGS. 15A-B is a set of graphs, showing that 4 clinical parameters and 2 genes allow for the identification of patients free of sAMR at one year post-transplantation. The forest plot of FIG. 15A summarizes the logistic regression model for sABMR-s. Values indicate odds ratios, and *, ** and *** represent p-values of <0.05, <0.01 and <0.001, respectively. The violin plots of FIG. 15B display sABMR-s values of sABMR compared to other patients with a Mann-Whitney p-value in the first cohort using qPCR (left) or NanoString (right). The dotted line indicates the optimal threshold (2.40 and 3.45 for qPCR and NanoString equations, respectively).

FIGS. 16A-C are a set of graphs demonstrating that the blood gene expression is independent of the measurement method. The two violin plots represent AKR1C3 expression (FIG. 16A) and TCL1A expression (FIG. 16B) in the sABMR group compared to others. AKR1C3 and TCL1A expression using individual qPCR compared to NanoString values are displayed in FIG. 16C. Gene expression is represented by the −ΔΔCt values for qPCR measures and log 2 of normalized counts for NanoString measures. p-values from Mann-Whitney's tests comparing sABMR to other are shown.

FIGS. 17A-D are a set of four violin plots, showing that immunosuppression treatment does not alter the sABMR-s discriminative ability. The violin plots display SABMR-s values of sABMR patients compared to others, depending on whether they take tacrolimus (FIG. 17A), corticosteroids (FIG. 17B), antiproliferative agents (FIG. 17C) or depletive induction treatment (FIG. 17D). The dotted line indicates the optimal threshold (2.40). p-values from Kruskal-Wallis with Dunn's post-hoc tests are shown.

### EXAMPLES

The present invention is further illustrated by the following examples.

### Example 1

### Materials and Methods

### Study Population

This non-interventional research project involved kidney transplant recipients followed up in the University Hospital of Nantes (France), Paris-Necker Hospital (France) and University Hospital of Lyon (France) for whom data were prospectively collected in the multicenter DIVAT database, approved by the French “National Commission on Informatics and Liberty” [CNIL] (DR-2025-087 No. 914184, Feb. 15, 2015) and the French Ministry of Higher Education and Research (file 13.334-cohort DIVAT RC12_0452, www.divat.fr).

For each patient, a blood sample in PAXgene™ tube (PreAnalytix, Qiagen, Hilden, Germany) was harvested at the time of a surveillance biopsy at one year after the transplantation. Samples were stored in the local Biological Resource Center (CRB) of the 3 participating hospitals and virtually mutualized on a common software (the CENTAURE biocollection declared since Aug. 13, 2008 to the Ministry of Research under No. PFS08-017; www.fondation-centaure.org). Each sample are in connection with the clinical data of the DIVAT database. Written consent was obtained for all patients. The clinical and research activities being reported are consistent with the Principles of the Declaration of Istanbul and in line with the good practice recommendations of the University Hospital of Nantes.

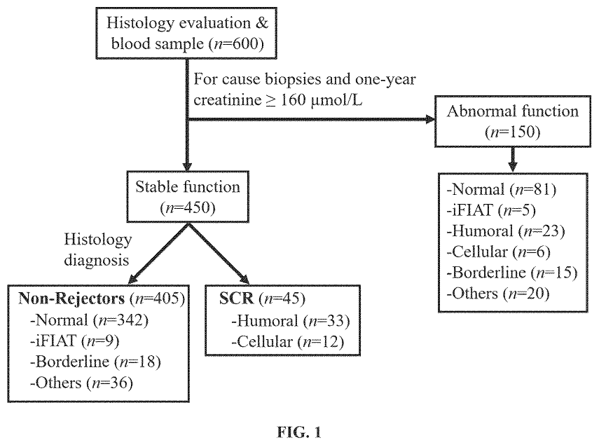

A total of 600 incident and consecutive patients met the inclusion criteria with paired PAXgene™ blood sample and surveillance biopsy at one-year post-transplantation. From these 600 patients, 450 displayed good function at one-year (serum creatinine levels below 160 μmol/L; mean eGFR (MDRD)=57.79±14.86 mL/min/1.73 m²) and protocol biopsies, and would benefit from non-invasive biomarker of SCR; while 150 patients displayed biopsy for indication and/or had one-year serum creatinine levels above 160 μmol/L (FIG. 1). Their clinical characteristics are summarized in Table 1: adults, kidney transplantation between January 2008 and January 2016, ABO-compatible, from heart beating or deceased donors. Patients with multiple organ transplants were not included. Available data included recipient characteristics (age, gender, history of diabetes, cardiovascular diseases and malignancy, the initial renal disease (recurrent or not), the renal replacement therapy and the cytomegalovirus (CMV) serology. Baseline transplantation parameters were transplantation rank, cold ischemia time, number of HLA-A-B-DR incompatibilities, pre-transplantation donor-specific antibodies (DSA), induction therapy (depleting versus non-depleting) and the delayed graft function (DGF, defined by the need of dialysis within the first week post-surgery). Donor features concerned age, gender, the donor type (living or deceased) and the last serum creatinine levels. Parameters collected in the first year after transplantation were the serum creatinine levels at 3- and 12-months post-transplantation, the rejections number, the maintenance treatments at 12 months (cyclosporine A (CsA), tacrolimus, mTORi, MMF/MPA, steroids) and the presence of de novo DSA at 12-months post-transplantation. The follow-up and data collection are stopped upon return to dialysis, death or re-transplantation.

TABLE 1
characteristics of the 450 transplanted patients Table 1 displays
clinical characteristics of the 450 patients who met inclusion
criteria, had paired protocol biopsy, blood RNA samples concomitant
with a normal function (serum creatinine levels < 160
umol/L) at one-year post-transplantation.

| | N | % |
|---|---|---|
| Male/Female recipients | 259/191 | 57.56/42.44 |

Allograft rank

| | | |
|---|---|---|
| First (i.e., no previous transplantation) | 382 | 84.89 |
| Second | 60 | 13.33 |
| Third | 7 | 1.56 |
| Fourth | 1 | 0.22 |

Initial disease

| | | |
|---|---|---|
| Undetermined etiology | 70 | 15.56 |
| Chronic glomerulonephritis | 122 | 27.11 |
| Chronic interstitial nephritis, urinary, | 191 | 42.44 |
| and others malformations | | |
| Vascular renal diseases | 31 | 6.89 |
| Diabetes | 36 | 8.00 |

Renal replacement therapy before transplantation

| | | |
|---|---|---|
| Preemptive | 85 | 18.89 |
| Transplantation | 54 | 12.00 |
| Peritoneal dialysis | 311 | 69.11 |
| Hemodialysis | | |
| History of diabetes | 68 | 15.11 |
| History of cardiovascular disease | 128 | 28.44 |
| History of malignancy | 43 | 9.56 |
| Positive recipient CMV serology | 271 | 60.22 |
| Positive donor CMV serology | 243 | 54.00 |
| Male/Female donor | 249/200 | 55.33/44.44 |
| Deceased/Live donor | 327/123 | 72.67/27.33 |

Deceased donor type

| | | |
|---|---|---|
| Non-beating heart | 21 | 4.67 |
| Beating heart | 306 | 68.00 |

HLA-A, -B and/or -DR mismatches

| | | |
|---|---|---|
| 0 | 23 | 5.11 |
| 1 | 23 | 5.11 |
| 2 | 79 | 17.56 |
| 3 | 118 | 26.22 |
| 4 | 126 | 28.00 |
| 5 | 56 | 12.44 |
| 6 | 24 | 5.33 |

Depleting induction

| | | |
|---|---|---|
| Non-depleting or none | 242 | 53.78 |
| Depleting | 208 | 46.22 |

Immunosuppression at 1 year

| CNIs | 420 | 93.33 |
|---|---|---|
| Cyclosporine A | 37 | 8.22 |
| Tacrolimus | 383 | 85.11 |
| mTOR inhibitor | 48 | 10.67 |
| Antiproliferative agents | 387 | 86.00 |
| Steroid | 335 | 74.44 |
| Reject before prelevement | 59 | 13.11 |
| Positive DSA pre-transplantation | 74 | 16.44 |
| Positive DSA at 1 year | 89 | 19.78 |
| | Mean | SD |
| Recipient age | 49.71 | 13.99 |
| Cold ischemic time (minutes) | 782.2 | 541.8 |
| 48ono rage (years) | 51.27 | 14.96 |
| Donor serum creatine levels (μmol/L) | 85.72 | 48.93 |
| 3-month eGFR (MDRD; mL/min/1.73 m²) | 56.28 | 17.71 |
| 1-year eGFR (MDRD; mL/min/1.73 m²) | 57.79 | 14.86 |

### Biopsy Evaluation

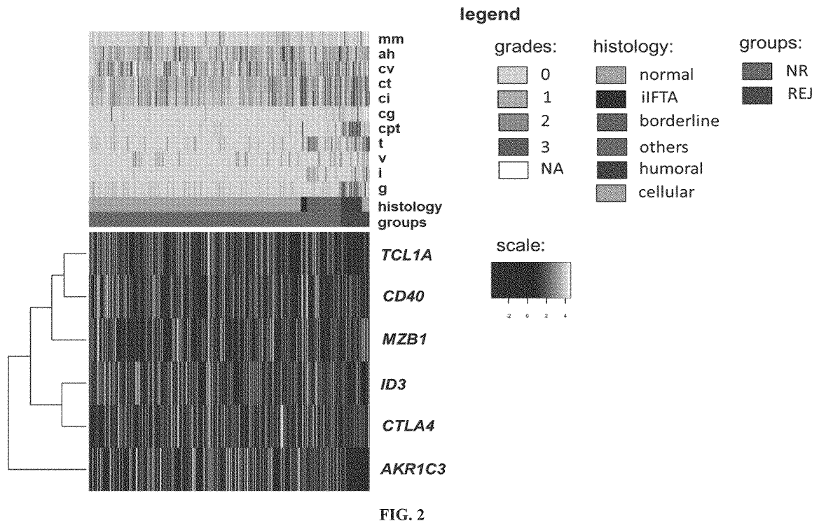

Kidney biopsies were interpreted and reviewed by a kidney pathologist at our institution, according to the 2015 Banff classification (Loupy et al., 2017. Am J Transplant. 17(1):28-41). The 450 protocol biopsies were categorized into two groups (FIG. 1 and FIG. 2):
(1) the SCR group (SCR, n=45): biopsies with evidences of SCR, either antibody (SABMR, n=33) or T-cell (sTCMR, n=12)-mediated rejections for which patients received treatment accordingly; and
(2) the Non-Rejector group (NR, n=405): biopsies with normal and subnormal histology biopsies exhibiting interstitial fibrosis and tubular atrophy (IFTA) and inflamed isolated IFTA grade 1 (i-IFTA), pooled and considered as normal biopsies (Normal, n=342); biopsies with inflamed IFTA of grade 2 and 3 (iFIAT, n=9), biopsies with borderline changes for whom patient have not been treated for (Bordeline, n=18), and biopsies with others lesions (Others, n=36) including recurrent (n=5) or de novo glomerulonephritis (n=3), BK virus nephropathy (n=2) and CNI toxicity (n=1).

### RNA Isolation

Peripheral blood samples were collected in PAXgene™ tubes (PreAnalytix), stored and shipped at −80° C. Total RNA extraction and purification were performed using the PAXgene™ Blood miRNA Kit (Qiagen, Hilden, Germany), according to the manufacturer's protocol, in the CRB of the University Hospital of Nantes. Total RNAs were quantified using a Nanodrop ND-1000 and 500 ng were used for real-time quantitative PCR (qPCR) and NanoString methods.

Gene Expression of the cSOT Score

For the qPCR, cDNA was synthesized using the High Capacity cDNA Reverse Transcription Kit (Thermo Fisher Scientific, Waltham; MA, USA). A TaqMan® Fast Advanced master mix was used with a StepOnePlus™ real-time PCR system (Thermo Fisher Scientific) using a final volume of 10 μL. Gene expression was assessed using commercially available TaqMan® probes (listed in Table 2) and normalized with the geometric mean of quantification cycle values (Cq) of B2M and HPRT1 references genes. Gene expression were run in duplicates and those with difference above 0.5 cycle or Cq above 35 were measured again and discarded if inconsistent. A commercial sample of total RNA from peripheral blood leukocytes (Takara Bio Europe SAS, Saint-Germain-en-Laye, France) was used as a calibrator to calculate gene expression according to the 2^{-ΔΔCt} method.

TABLE 2
Genes analyzed with NanoString and TaqMan ® information.
TaqMan ® probe references (Thermo Fisher Scientific)
and NanoString-targeted sequences are displayed for the 13 measured genes.

| | | | Reference | NanoString |
|---|---|---|---|---|
| Gene Symbol | Accession number | Taqman probe | genes | targeted sequence |
| ActB | NM_001101.3 | | Yes | SEQ ID NO: 1 |
| AKR1C3 | NM_001253908.1 | Hs00366267_m1 | | SEQ ID NO: 2 |
| B2M | NM_004048.2 | Hs00984230_ml | Yes | SEQ ID NO: 3 |
| CD40 | NM_001250.5 | Hs00374176_m1 | | SEQ ID NO: 4 |
| CTLA4 | NM_001037631.2 | Hs00175480_ml | | SEQ ID NO: 5 |
| GAPDH | NM_001289746.1 | | Yes | SEQ ID NO: 6 |
| HPRT1 | NM_000194.2 | Hs99999909_ml | Yes | SEQ ID NO: 7 |
| ID3 | NM_002167.4 | Hs00171409_ml | | SEQ ID NO: 8 |
| MS4A1 | NM_152866.2 | | | SEQ ID NO: 9 |
| MZB1 | NM_016459.3 | Hs00414907_ml | | SEQ ID NO: 10 |
| TCL1A | NM_001098725.1 | Hs00951350_ml | | SEQ ID NO: 11 |
| TUBA4A | NM_006000.1 | | Yes | SEQ ID NO: 12 |
| YWHAZ | NM_003406.2 | | Yes | SEQ ID NO: 13 |

The NanoString PlexSet™ Technology (NanoString Technologies, Seattle, WA, USA) was used to measure 6 genes (AKR1C3, CD40, CTLA4, ID3, MZB1 and TCL1A) plus 6 references genes, according to the manufacturer's instructions. The MS4A1 gene (coding for CD20) was measured in parallel.

These six genes were selected based on previous works by the Inventors (WO 2018/015551; Danger et al., 2017. Kidney Int. 91(6):1473-1481), which describe a composite score (cSoT) associated with spontaneous operational tolerance in kidney transplant recipients (i.e., stable and acceptable graft function without immunosuppression for years), comprising the expression levels of these six genes and two clinical parameters (age of the transplanted patient at transplantation time and at blood sampling time).

Capture probes for the genes of interest were designed by NanoString support and synthesized by Integrated DNA Technologies (IDT, Coralville, IA). According titration experiments, a total RNA input of 500 ng was chosen and 99% attenuation signal of 3 highly expressed genes (ACT, B2M, GAPDH) was performed using unlabeled probes, not to saturate cartridge signal, as recommended by the provider. Calibration between 2 lots of reagents was performed by NanoString support with the common calibrator samples used for qPCR. ID3 values were discarded as they were below the expression threshold calculated as:

2×(mean of negative controls+(2×standard deviation of negative controls))

The geometric mean of the six reference genes (ACT, B2M, GAPDH, HPRT1, TUBA4A and YWHAZ) was used to normalize gene expression using the NanoString nSolver™ software 4.0. Samples with bad quality controls and values below the expression threshold were discarded. For replicated samples, mean of expression values were calculated if correlation was above 0.95. Log2 of normalized counts or −ΔΔCt was used for downstream analysis for NanoString and qPCR values, respectively.

### Statistical Analysis

Comparisons of two groups were performed using the Student's 1-test with more than 30 samples, while multiple group comparisons were performed using non-parametric Kruskal-Wallis with Dunn's post-hoc tests for continuous variables and using χ² test or Fisher's exact test for categorical variables. Pearson correlation was used to assess relationship between continuous data. Logistic regression was built to assess relationship between histological groups and explanatory variables using stepwise regression and compared using the Akaike information criterion (AIC). Absence of lack of fit was assess using unweighted sum of squares test (Hosmer et al., 1997. Stat Med. 16(9):965-80) (using the package rms). Area under the receiver operating characteristics (ROC) curve (AUC), with a 95%-confidence interval (CI), was used to evaluate model performances and ROC curves comparison was performed using bootstrap test (n=1000) with the same number of controls and cases than the original sample (package pROC) (Robin et al., 2011. BMC Bioinformatics. 12:77). Internal validation was performed by bootstrapping (n=1000) (Steyerberg et al., 2001. J Clin Epidemiol. 54(8): 774-81) using the caret package. Multiple testing was corrected with Benjamini-Hochberg correction when appropriated. Analyses were performed using R 4.0.3 and GraphPad Prism v.9 (GraphPad Software, La Jolla, CA, USA).

### Results

### Demographic Description of the Cohort of Transplanted Patients

Clinical characteristics of the 450 kidney transplanted patients who met inclusion criteria, had paired protocol biopsy and blood RNA samples at one-year post-transplantation and with serum creatinine levels below 160 μmol/L are summarized in Table 1. Patients received standard maintenance immunosuppressive therapy, mostly calcineurin inhibitors (CNIs: 93.3%; primarily tacrolimus: 85.1%), antiproliferative agents (86.0%; including mycophenolate mofetil (MMF), mycophenolic acid (MPA) or azathioprine), and a corticosteroid regimen (74.4%). 74 patients (16.4%) had anti-HLA DSA at the time of transplantation and 89 (19.78%) displayed DSA one-year after transplantation.

The cSoT Score is Decreased in Patients with SCR at One-Year Post-Transplantation

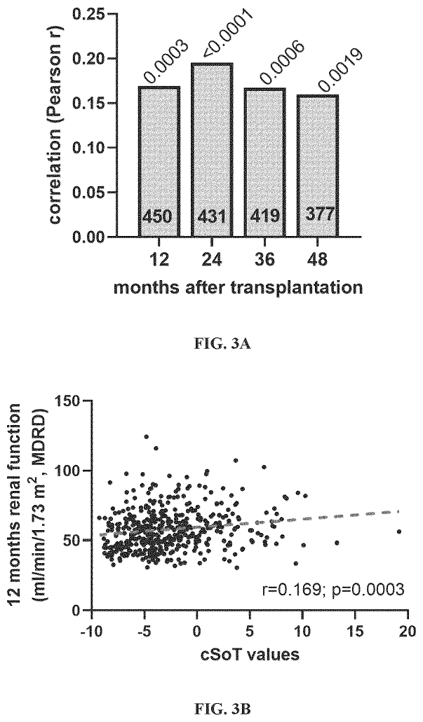

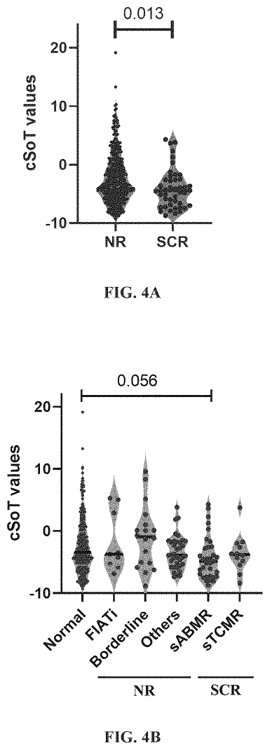

At one-year post-transplantation, among the 450 patients with good function at one-year (serum creatinine levels below 160 μmol/L), we found that the cSoT score (described in WO 2018/015551 and Danger et al., 2017. Kidney Int. 91(6):1473-1481) was significantly associated with renal function (MDRD) at 12-, 24-, 36- and 48-month post-transplantation (p<0.01) (FIGS. 3A and 3B). According to this classification, we found that the cSoT score was significantly decreased in blood from SCR patients compared to NR patients (adj. p=0.013; FIGS. 4A and 4B) and exhibited a ROC AUC of 0.615 (95% CI=[0.530-0.700]).

From the cSoT Score, AKR1C3 and TCL1A are Sufficient to Diagnose Patients Unlikely to Display SCR

We first tested the six genes and the two clinical parameters composing the cSoT score independently and in association for their capacity to diagnose SCR in the 450 patients with normal renal function. We first show that the two clinical parameters (recipient age at transplantation and at blood sampling time) were not significantly differential in SCR patients compared to NR patients (p=0.932 and 0.936, respectively) and had thus no impact on the ability of the cSoT score to discriminate the patients.

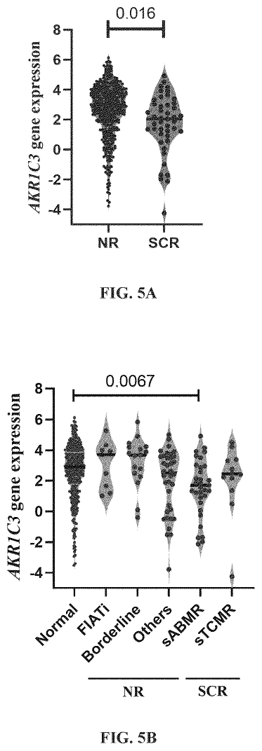

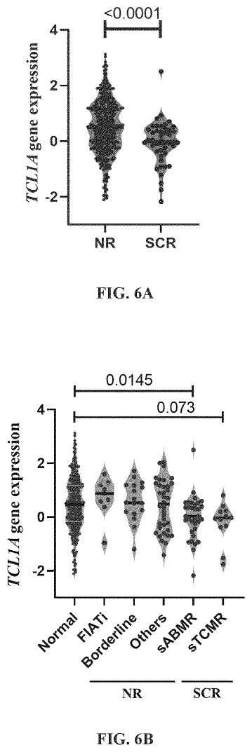

Among the six genes, only AKR1C3 and TCL1A expression were significantly decreased in blood from SCR patients compared to NR patients (adj. p=0.016 and <0.0001, respectively) (FIGS. 5A and 5B for AKR1C3; FIGS. 6A and 6B for TCL1A), with a mean decrease in the 45 patients of the SCR groups of 45.9% for AKR1C3 and 81.3% for TCL1A.

When used alone, AKR1C3 and TCL1A allowed to discriminate SCR patients with an AUCs of 0.623 (95% CI=[0.604-0.741]) and 0.640 (95% CI=[0.558-0.721]), respectively. When associated together, these two genes allowed even higher and good discrimination of SCR patients with an AUCs of 0.703 (95% CI=[0.629-0.777]).

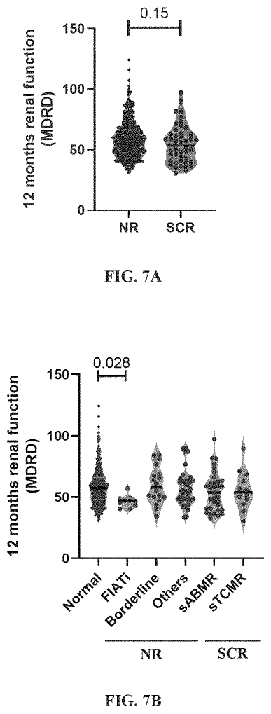

We then investigated histologic diagnostic of these 450 patients. Both genes were significantly decreased in patients with sABMR compared to patients with normal histology (p=0.0067 and p=0.0145 for AKR1C3 and TCL1A, respectively). A trend for a decrease of AKR1C3 was also observed in patients with sTCMR compared to patients with normal histology (p=0.073) (FIG. 5B). The association of the two genes even reinforces the difference between sABMR and normal histology (p=0.0002). By comparison, the renal function at one-year post-transplantation was not different between normal histology and either of sABMR or sTCMR (FIGS. 7A and 7B).

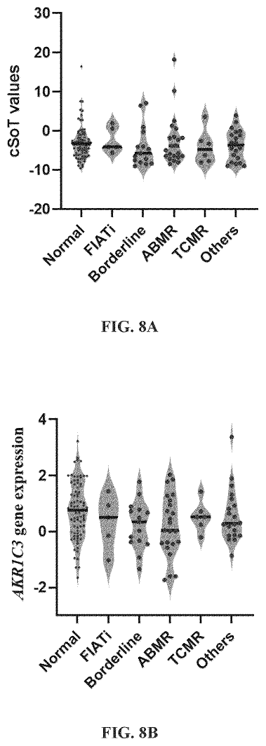

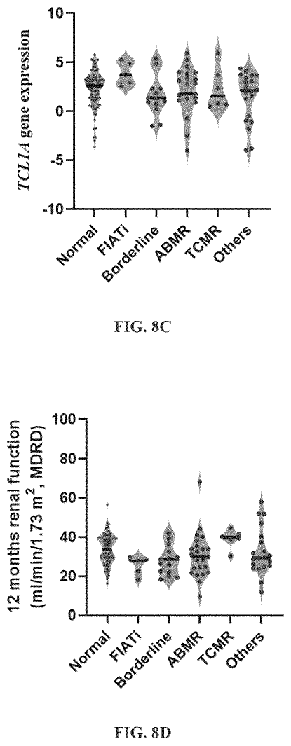

Finally, no difference neither for AKR1C3 nor for TCL1A was observed between the different histological groups within the 150 patients with abnormal function and/or for-cause biopsy evaluation (FIG. 8A-D).

### A New Composite Model Allows Identifying SCR-Free Patients at One-Year Post Transplantation

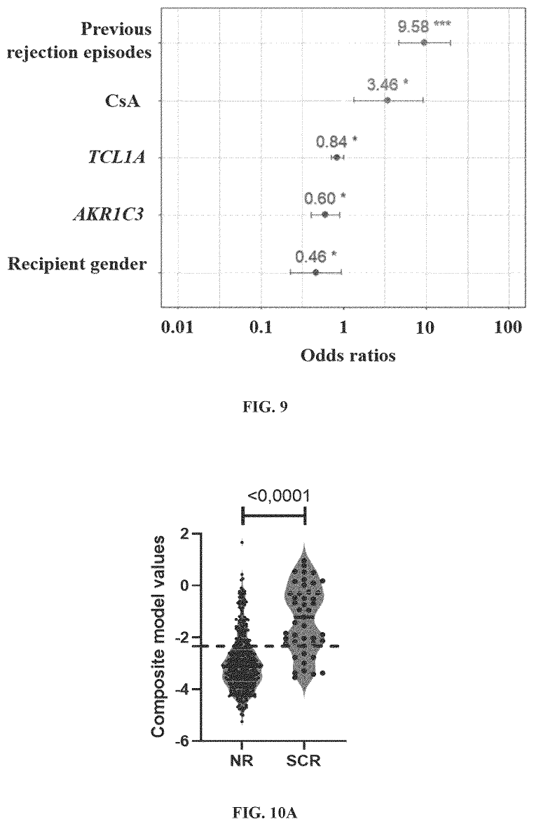

The clinical parameters significantly associated with SCR compared to NR in a univariate analysis were determined to be the experience of rejection episodes before blood sampling (p<0.0001), the presence of DSA before transplantation and at blood sampling time (p<0.001 and p=0.0025, respectively), the recipient gender (p=0.0057), the uptake of corticosteroids and CsA (p=0.012 and p=0.018, respectively) (Table 3). Only the experience of rejection episodes before blood sampling, the recipient gender and the uptake of CsA at blood sampling time were retained as significantly associated with SCR in the multivariate analysis (FIG. 9 and Table 3).

TABLE 3
univariate and multivariate logistic regression
analysis of clinical parameters for SCR diagnosis

| | | | Univariate analysis | | Multivariate analysis | |
|---|---|---|---|---|---|---|
| | Number of cases (%) | | | OR | | OR |
| | NR | SCR | p | (95% CI) | p | (95% CI) |
| Experience of | 36 | 23 | 6.73 × | 10.72 | 2.90 × | 10.80 |
| rejection episodes | (8.89) | (48.89) | 10⁻¹² | (5.45-21.26) | 10⁻¹¹ | (5.38-22.03) |
| DSA before | 58 | 16 | 0.000656 | 3.28 | | |
| transplantation | (15.8) | (38.1) | | (1.63-6.44) | | |
| DSA at blood | 72 | 17 | 0.00247 | 2.81 | | |
| sampling | (19.5) | (40.5) | | (1.42-5.44) | | |
| Recipient gender | 242 | 17 | 0.00574 | 0.41 | 0.01566 | 0.43 |
| (M/F) | (59.8) | (37.8) | | (0.21-0.76) | | (0.21-0.84) |
| Corticosteroids | 292 | 41 | 0.0121 | 3.84 | | |
| uptake | (72.8) | (91.1) | | (1.50-12.98) | | |
| CsA uptake | 29 | 8 | 0.0178 | 2.80 | 0.00611 | 3.77 |
| | (7.2) | (17.8) | | (1.13-6.34) | | (1.39-9.47) |
| Tacrolimus | 348 | 31 | 0.00089 | 0.30 | 0.00028* | 0.218* |
| uptake | (87.88) | (68.89) | | (0.15-0.51) | | (0.095-0.50) |

OR: odds ratio
*values in an alternative score, with tacrolimus uptake instead of CsA uptake.

We then performed a composite model based on the expression of the two genes, AKR1C3 and TCL1A, and these three clinical variables and tested its discriminative power on the 450 patients with normal graft function. This score (referred to as SCR-s) was built using a logistic regression, where the experience of rejection episodes and CsA uptake were positively associated with the risk of SCR, while the recipient gender (male versus female recipient), TCL1A and AKR1C3 expression were negatively associated with the risk of SCR (likelihood ratio p<0.0001; FIG. 9). Alternatively, we can define the experience of rejection episodes and CsA uptake to be negatively associated with NR status, while the recipient gender (male versus female recipient), TCL1A and AKR1C3 expression are positively associated with NR status.

In an alternative score, rather than taking CsA uptake into account, this parameter was replaced with the uptake of another immunosuppressant, tacrolimus, far more used in clinical practice than cyclosporin A. In the SCR-s, tacrolimus uptake was negatively associated with the risk of SCR (or alternatively, was positively associated with NR status). This difference between CsA uptake and tacrolimus uptake in the SCR-s is self-explanatory: when the patient takes CsA, they typically do not take tacrolimus and this parameter is positively associated with the risk of SCR; conversely, when the patient takes tacrolimus, they typically do not take CsA and this parameter is negatively associated with the risk of SCR.

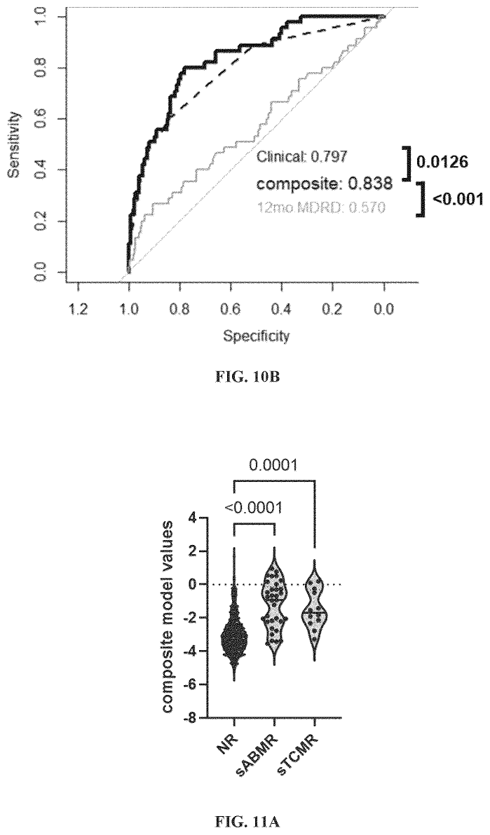

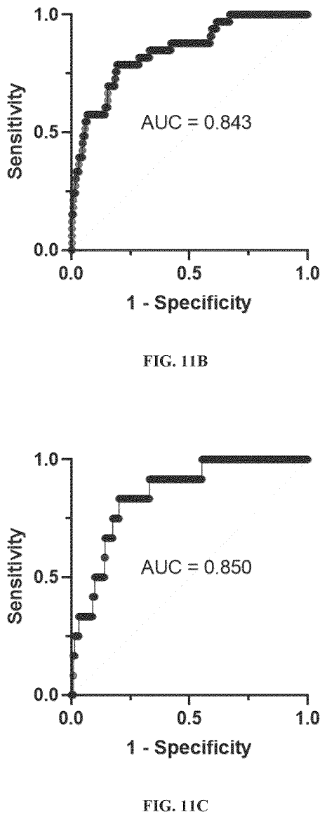

This SCR-s was significantly higher (mean increase of 67.70% in the 45 patients of the SCR groups) in the SCR group compared to the NR group (p<0.0001; FIG. 10A) and displayed high discriminative ability with an AUC of 0.838 (95% CI=[0.779-0.897]), significantly higher than clinical parameters only (AUC=0.797 (95% CI=[0.726-0.867]); p=0.0126; FIG. 10B). Furthermore, the SCR-s exhibited significant higher values in sABMR or sTCMR compared to NR (p<0.0001) and similar AUCs when tested independently on both groups (AUC=0.843 (95% CI=[0.773-0.914] and 0.850 (95% CI=[0.761-0.939] for sABMR and sTCMR, respectively; FIG. 11A-C). At the optimal threshold (Youden's index), specificity and sensibility were respectively of 0.78 and 0.80, with 317 patients identified as true negative out of the 405 NR patients and 36 identified as true positive out of the 45 SCR patients (FIG. 10A). At such threshold, the negative predictive value (NPV) was 97.2% and positive predictive value (PPV) was 29.0%. Finally, internal validation using a bootstrapping resampling (n=1000) to correct model optimism allowed high performance with an AUC of 0.810 (95% CI=[0.73-0.89]).

### Validation of the SCR-s on an Independent Platform

The SCR-s was measured using the enzyme-free and probes hybridization-based NanoString method with different probes than the standard qPCR used beforehand. We found a high and significant correlations between qPCR and NanoString gene expression (r=0.92 and 0.778 for TCL1A and AKR1C3, respectively; p<0.001) (Table 4).

TABLE 4
Blood gene expression is independent of measurement method

| | | NanoString | | | | |
|---|---|---|---|---|---|---|
| | | AKR1C3 | CD40 | CTLA4 | MZB1 | TCL1A |
| Individual | AKR1C3 | 0.77 | −0.19 | 0.08 | −0.05 | −0.04 |
| qPCR | CD40 | −0.22 | 0.7 | 0.05 | 0.5 | 0.61 |
| | CTLA4 | 0.04 | 0.23 | 0.67 | 0.33 | 0.17 |
| | MZB1 | −0.15 | 0.33 | 0.11 | 0.81 | 0.42 |
| | TCL1A | −0.06 | 0.53 | 0.05 | 0.57 | 0.93 |

The correlogram represent r Pearson correlation (from 1 to −1) of gene expression between qPCR and NanoString measures from the 450 patients.

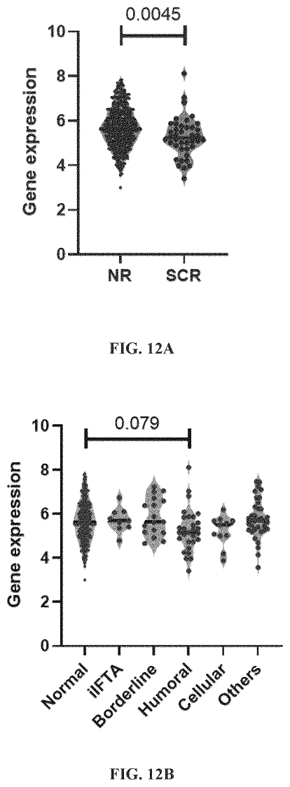

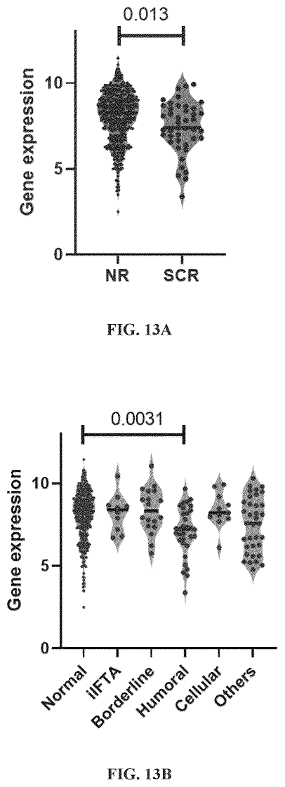

Using the enzyme-free and probes hybridization-based NanoString method, we confirmed the significant down-regulation of AKR1C3 and TCL1A in SCR patients compared to NR patients (p=0.0045 and 0.013, respectively) (FIG. 12A-B for AKR1C3 and FIG. 13A-B for TCL1A) and the ability of the SCR-s to discriminate patients with SCR with an AUC of 0.815 (95% CI=[0.728-0.880]).

### Validation of the SCR-s in an Independent and Multicenter Validation Set

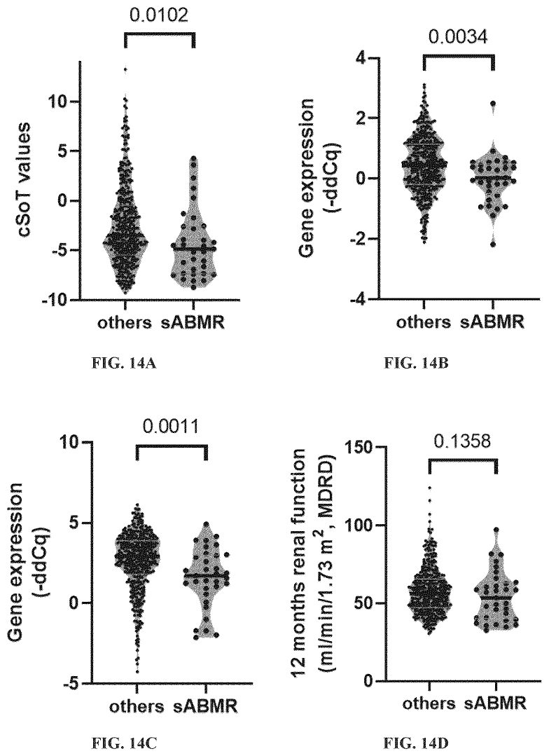

The validation set included 110 patients, including 11 patients with SCR. In this cohort, the established SCR-s allowed a correct classification of 9 out of 11 SCR patients, resulting in an AUC of 0.884 (95% CI=[0.701-0.99]) (FIG. 14).

Using the optimal threshold determined in the training set (Youden's index), the specificity and sensitivity were 0.798 and 0.909, respectively, with an NPV of 98.7%.

### DISCUSSION

SCR is detected only on protocol biopsies from patients with normal allograft function and affects up to 25% of renal biopsies at 1-year post-transplantation with an incidence that is inversely correlated to the time post-transplant (Couvrat-Desvergnes et al., 2019. Nephrol Dial Transplant. 34(4): 703-711; Loupy et al., 2015. J Am Soc Nephrol. 26(7): 1721-31; Nankivell et al., 2004. Transplantation. 78(2):242-9).

Detection of such lesions is associated with unfavorable outcomes (Filippone & Farber, 2020. Transplantation; Loupy et al., 2015. J Am Soc Nephrol. 26(7): 1721-31; Mehta et al., 2017. Clin Transplant. 31(5); Nankivell et al., 2004. Rush & Gibson, Transplantation. 78(2):242-9; 2019. Transplantation. 103(6):e139-e145; Shishido et al., 2003. J Am Soc Nephrol. 14(4): 1046-52) and early treatment is beneficial for graft outcome (Kee et al., 2006. Transplantation. 82(1):36-42; Parajuli et al., 2019. Transplantation. 103(8): 1722-1729; Rush et al., 1998. J Am Soc Nephrol. 9(11):2129-34). Thus, detection of such insidious lesions using non-invasive diagnostic tools would improve graft outcome, while diagnosing SCR-free patients could also help avoiding invasive procedures on patients with no serious histological lesions and improve patient management (Couvrat-Desvergnes et al., 2019. Nephrol Dial Transplant. 34(4): 703-711; Friedewald & Abecassis, 2019. Am J Transplant. 19(7):2141-2142).

We previously reported a 6-gene blood signature that allows detecting patients with operational tolerance (Brouard et al., 2012. Am J Transplant. 12(12):3296-307; Danger et al., 2017. Kidney Int. 91(6): 1473-1481; WO 2018/015551). Furthermore, this score was found to be decreased in patients who developed anti-HLA antibodies, suggesting its association with immune tolerance loss.

Based on these data, we hypothesized that this score would be an ideal signature of low risk of immunological rejection and we tested its ability to diagnose SCR in patients with stable graft function early after transplantation. Herein, we showed that, not only is this score associated with SCR, but we improved it.

We showed that only two genes, AKR1C3 and TCL1A, allow independently from each other to identify patients affected with SCR; and that the combination of both these genes allows an even better discrimination.

We then build a composite score (SCR-s) based on expression of these two genes and 3 clinical variables (experience of previous rejection episodes, immunosuppressant uptake [in particular CsA uptake or tacrolimus uptake] and recipient gender), that allows detecting absence of SCR with high power at one-year post-transplantation.

Several biomarkers of SCR have previously been proposed, including blood gene signatures (WO 2015/179777; WO 2019/217910; Crespo et al., 2017. Transplantation. 101(6): 1400-1409; Friedewald et al., 2019. Am J Transplant. 19(1):98-109; Van Loon et al., 2019. EbioMedicine. 46:463-472; Zhang et al., 2019. J Am Soc Nephrol. 30(8): 1481-1494). Zhang published a signature of 17 genes able to diagnose SCR and acute cellular rejection with an 89% NPV and a 73% PPV at 3-month post-transplantation (Zhang et al., 2019. J Am Soc Nephrol. 30(8): 1481-1494). Similarly, a signature of 51 genes allows identifying SCR at 24-month post-transplantation (Friedewald et al., 2019. Am J Transplant. 19(1):98-109). These signatures did not include AKR1C3 and TCL1A, which may be explained by the fact that in both studies, mainly cellular and borderline rejections were analyzed. Van Loon reported on a signature of 8 genes, but only to diagnose ABMR, with performances comparable to our SCR-s for sABMR (Van Loon et al., 2019. EbioMedicine. 46:463-472). Finally, the 17-genes signature of the kSort study has also been proposed to diagnose 6-month SABMR (Crespo et al., 2017. Transplantation. 101(6): 1400-1409) but was not validated in a large cohort of 1134 patients (Van Loon et al., 2021. Am J Transplant. 21(2): 740-750).

We report here a composite score (SCR-s) with only two genes and three clinical parameters that allows detecting SCR-free patients with normal graft function with a single blood sample at one-year post-transplantation. This non-invasive tool may be used to avoid biopsy for patients unlikely to display SCR among the large population of kidney transplanted recipients. Indeed, this SCR-s reaches a 97.2% NPV, meaning that a negative test is a true negative with high probability. As an example, this would result in our cohort of 450 patients in the avoidance of 317 biopsies. Moreover, and contrary to the prior solutions detailed above, our SCR-s allowed to detect both subclinical T-cell mediated rejection (sTCMR) and sABMR. This SCR-s may be easily implemented in routine, using qPCR largely available in clinical centers, unlike for microarrays- or RNA sequencing-based signatures. We also validated the model using both classical qPCR and NanoString platforms, reinforcing its technical robustness and its cost effectiveness.

We performed a first validation of the SCR-s on an independent and multicenter validation set including 110 patients (11 patients diagnosed with SCR during surveillance biopsy): the SCR-s offered a correct classification for 9 out of 11 SCR patients.

Despite our model needing further validation on independent cohorts of patients, our hypothesis-driven study was focused on the measure of only very few genes, thus reducing the “by-chance” association of parameters that could arise in fishing studies. Furthermore, our analysis has been done in a “real-life” scenario, without prior patient selection on a large cohort of patients.

### Example 2

While the SCR-s of Example 1 has allowed us to diagnose SCR, whether T-cell mediated rejection (sTCMR) and subclinical antibody-mediated rejection (sABMR), we aimed at developing an alternative composite model which would be specific of subclinical antibody-mediated rejection (sABMR) only.

### Materials and Methods

Same as in Example 1.

### Results

Identification of Genes and Clinical Parameters Associated with sABMR

Among the kidney transplanted patients of the study cohort who met inclusion criteria, we selected 33 with biopsy-proven sABMR (SCR “humoral” on FIG. 1).

We found that the cSoT (described in WO 2018/015551 and Danger et al., 2017. Kidney Int. 91(6): 1473-1481) was significantly decreased in blood from these patients from the sABMR group as compared to patients from the NR group and to patients with STCMR (p=0.0102; FIG. 14A), with an AUC of 0.578 (95% CI=[0.465-0.691]).

From the 6 genes composing the cSoT, the expression levels of AKR1C3 and TCL1A were significantly decreased in blood from these 33 patients with sABMR compared to all the other patients (p=0.0034 and 0.0011, respectively) (FIGS. 14B and 14C). When used alone, AKR1C3 and TCL1A allowed the discrimination of patients with SABMR with AUCs of 0.652 (95% CI=[0.570-0.734]) and 0.669 (95% CI=[0.578-0.760]), respectively. When combined, the 2 genes allowed a good discrimination of patients with sABMR with an AUC of 0.711 (95% CI=[0.624-0.797]) while renal function did not significantly differ between the 2 groups (p=0.136) (FIG. 14D).

Eleven clinical parameters were significantly associated with sABMR in univariate analysis (p<0.20) including experience of rejection episodes before blood sampling (p<0.0001), allograft rank (p<0.0001), use of a deleting induction treatment (p=0.00235), recipient's gender (p=0.00477), recipient CMV positivity (p=0.0279), corticosteroid uptake at 12 months post-transplantation (p=0.0318), and a number of HLA-A, -B and -DR incompatibilities between donor and recipients strictly greater than 3 (p=0.0362).

Building a Score for the Detection of sABMR (SABMR-s)

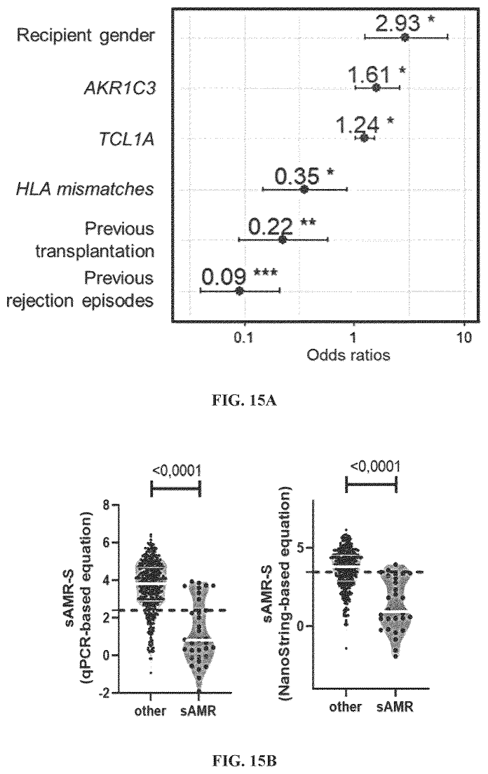

From these 11 clinical parameters and the 2 genes significantly associated with sABMR in the univariate analysis, a refined composite score of sABMR (sABMR-s) was built using a multivariate logistic regression with stepwise selection and bootsraping resampling: experience of rejection episodes before blood sampling, allograft rank and HLA mismatches were positively associated with sABMR status, while TCL1A and AKR1C3 blood gene expression and recipient's gender were negatively associated with sABMR status in this sABMR-s (FIG. 15A). The sABMR-s was significantly lower in the sABMR group than in the group with other diagnosis (NR group and sTCMR group) (p<0.0001; FIG. 15B) and displayed high discriminative ability with an AUC of 0.860 (95% CI=[0.794-0.925]).

While the presence of donor-specific antibodies (DSA) during the first year post-transplantation was significantly associated with sABMR in the univariate analysis (p<0.0001), this parameter did not allow to discriminate sABMR significantly better than the 2 genes together (AUC=0.768 (95% CI=[0.686-0.849]), p=0.307).

In order to build a sABMR score independent of DSA measure, and according to the high prevalence of de novo DSA (dnDSA)-positive patients who are free of sABMR lesions (64 patients in our cohort and 60% according the literature; see., e.g., Yamamoto et al., 2016. Transplantation. 100(10):2194-2202, or Bertrand et al., 2020. Transplantation. 104(8):1726-1737), DSA experience was not used for the sABMR-s construction. Our sABMR-S was more discriminative than DSA experience in the first year of transplantation (p=0.00923) and addition of DSA experience in the sABMR-s did not significantly improve its discriminative performance (AUC=0.877; 95% CI=[0.809-0.944]); p=0.222). Interestingly, the sABMR-s remained significantly decreased in sABMR patients compared to DSA-positive patients with other diagnosis than sABMR (p=0.0011) and with an AUC of 0.77 (95% CI=[0.666-0.875]).

In addition, within the 147 patients that had a biopsy for medical indication, the SABMR-s was also significantly decreased for the 23 patients with sABMR, and for cause biopsy compared to the 124 patients with for cause biopsies with other diagnosis (p=0.0023).

At the optimal threshold maximizing specificity and sensitivity (Youden's index), corresponding to a value of 2.40, the sABMR-s displayed a specificity and sensitivity of 0.840 and 0.758, respectively. At this threshold, the sABMR-s had a negative predictive value (NPV) of 97.7% and a positive predictive value (PPV) of 27.7%, with 342 patients identified as true negative out of 408 patients with other diagnosis than sABMR (83.8%) and 25 identified as true positive out of the 33 sABMR patients (75.8%). Finally, internal validation using a bootstrapping resampling (n=1000) to correct model optimism allowed high performance with an AUC of 0.830 (95% CI=[0.74-0.92]).

Validation of sABMR-s on an Independent Technical Platform

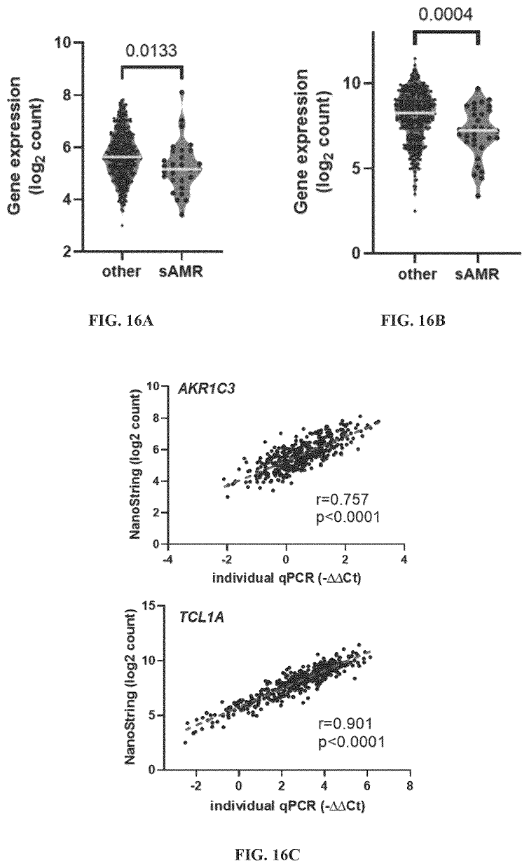

The sABMR-s was built using the standard qPCR method for AKR1C3 and TCL1A measures. To allow its use at a large scale, we validated the technique using the enzyme-free and probe hybridization-based NanoString platform with different probes than those used for qPCR. We confirmed the significant downregulation of AKR1C3 and TCL1A in sABMR compared to the other groups (p=0.013 and 0.0004, respectively) (FIG. 16A-B) with high and significant correlations between qPCR and NanoString gene expression (r=0.901 and 0.757 for TCL1A and AKR1C3, respectively, p<0.0001) (FIG. 16C). Since qPCR and NanoString measures exhibited different dynamic ranges, we used the sABMR-s parameters and adjusted coefficients with the NanoString data. The discrimination ability of the sABMR-s with NanoString data reached similar discriminative ability than with qPCR with an AUC of 0.859 (95% CI=[0.793-0.925]) (p<0.0001; FIG. 15B).

Immunosuppression Did not Alter the Discriminative Power of the sABMR-s

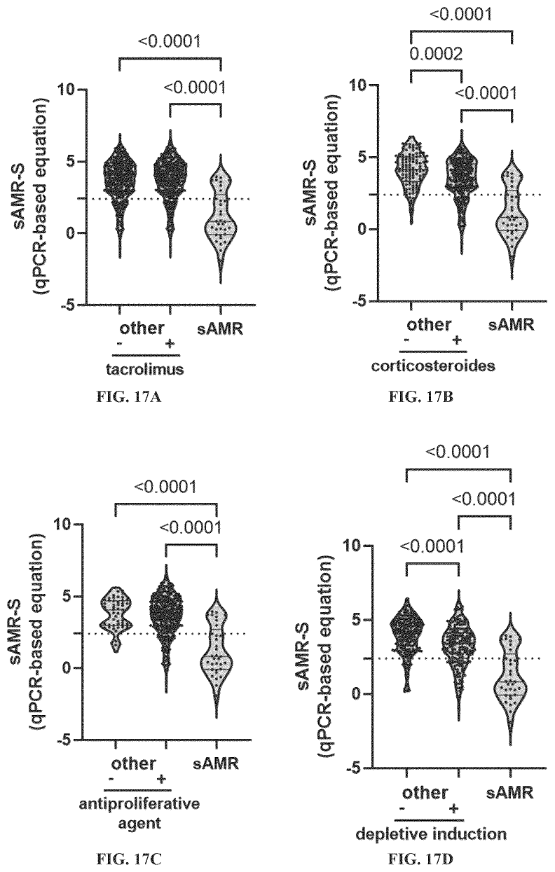

The sABMR-s was slightly decreased in patients with no lesion of sABMR treated either with corticosteroids or with anti-thymocyte globulin (ATG)-depleting induction treatment compared to those with no lesion of sABMR without corticosteroids or treated with non-depleting treatment or receiving no induction therapy (p=0.0002 and <0.0001, respectively). In subsets of patients treated with tacrolimus (FIG. 17A), corticosteroids (FIG. 17B), antiproliferative agents (FIG. 17C) or depletive induction treatment (FIG. 17D), values of AUC comparing sABMR to others were of 0.864 (95% CI=[0.802-0.926]), 0.839 (95% CI=[0.767-0.911]), 0.855 (95% CI=[0.790-0.921]) and 0.811 (95% CI=[0.726-0.896]), respectively. Thus, the sABMR-s remained able to discriminate patients with and without sABMR lesions irrespective of their treatment.

1-29. (canceled)

30. A method of treating subclinical kidney rejection in a subject in need thereof, comprising the steps of:
1) diagnosis subclinical kidney rejection in the subject by:
a) determining the level, amount or concentration of at least one biomarker selected from the group consisting of TCL1A and AKR1C3 in a sample previously taken from the subject;
b) comparing the level, amount or concentration of the at least one biomarker with the level, amount or concentration of the same at least one biomarker determined in at least one reference subject,
wherein the at least one reference subject is:
a subject who has not undergone kidney transplantation,
a kidney transplant recipient who is not affected with subclinical kidney rejection, or
the subject investigated for subclinical kidney rejection themselves prior to kidney transplantation; and
c) concluding that the subject is affected with subclinical kidney rejection when the level, amount or concentration of the at least one biomarker is statistically significantly lower than the level, amount or concentration of the same at least one biomarker determined in the at least one reference subject, and
2) treating the subject being diagnosed with subclinical rejection during the first step of the method.

31. The method according to claim 30, wherein step a) comprises determining the level, amount or concentration of TCL1A in the sample previously taken from the subject.

32. The method according to claim 30, wherein step a) comprises determining the level, amount or concentration of AKR1C3 in the sample previously taken from the subject.

33. The method according to claim 30, wherein step a) comprises determining the level, amount or concentration of both TCL1A and AKR1C3 in the sample previously taken from the subject.

34. The method according to claim 30, wherein the level, amount or concentration of the at least one biomarker is expressed in terms of absolute or relative levels, amounts or concentrations.

35. The method according to claim 30, wherein step 1) comprises:
a) determining a composite score with the level, amount or concentration of the at least one biomarker selected from the group consisting of TCL1A and AKR1C3, wherein said composite score is established using Formula (1):
$$composite score = \sum {β}_{i} {X}_{i} + {β}_{0} \qquad ( 1 )$$
wherein:
“βᵢ” represents the regression coefficient for the level, amount or concentration of each of the at least one biomarker;
“Xᵢ” represent the predictor variable for the level, amount or concentration of each of the at least one biomarker;
“β₀” represents the intercept of the equation,
b) comparing the composite score with a reference composite score determined in the at least one reference subject;
c) concluding that the subject is affected with subclinical kidney rejection when the composite score is substantially higher than the reference composite score determined in the at least one reference subject.

36. The method according to claim 30, wherein step 1) comprises:
a) determining a composite score, with:
the level, amount or concentration of the at least one biomarker selected from the group consisting of TCL1A and AKR1C3; and
one, two, or three clinical parameters selected among:
the experience of rejection episodes before blood sampling,
the recipient gender, and
the uptake of immunosuppressant (IS) at blood sampling,
wherein said composite score is established using Formula (2):
$$composite score = \sum ( {β}_{previous rejection episode} \times previous rejection episode + {β}_{IS uptake} \times IS uptake + {β}_{recipient gender} \times recipient gender + {β}_{TCL 1 A} \times Expr ( TCL 1 A ) + {β}_{AKR 1 C 3} \times Expr ( AKR 1 C 3 ) ) + {β}_{0} \qquad ( 2 )$$
wherein:
“β_{TCL1A}”, “β_{AKR1C3}”, “β_{previous rejection episode}”, “β_{IS uptake}”, and “β_{recipient gender}” represent the regression coefficients for each predictor among the level, amount or concentration of the biomarkers and the clinical parameters;
“previous rejection episode” represents the predictor variable defining the experience of rejection episodes before blood sampling, with 0=“no previous rejection episodes” and 1=“one or several previous rejection episodes”;
“IS uptake” represents the predictor variable defining the uptake of immunosuppressant (IS) selected from tacrolimus or cyclosporine A (CsA) at blood sampling time, with 0=“no CsA uptake” or “tacrolimus uptake”, and 1=“CsA uptake” or “no tacrolimus uptake”;
“recipient gender” represents the predictor variable defining the gender of the graft recipient, with 0=“female” and 1=“male”;
“Expr(TCL1A)” and “Expr(AKR1C3)” represent the predictor variables defining the level, amount or concentration of TCL1A and AKR1C3, respectively; and
“β₀” represents the intercept of the equation;
b) comparing the composite score with a reference composite score determined in the at least one reference subject;
c) concluding that the subject is affected with subclinical kidney rejection when the composite score is substantially higher than the reference composite score determined in the at least one reference subject.

37. The method according to claim 36, wherein the uptake of immunosuppressant (IS) at blood sampling is the uptake of tacrolimus at blood sampling.

38. The method according to claim 36, wherein the uptake of immunosuppressant (IS) at blood sampling is the uptake of cyclosporine A (CsA) at blood sampling.

39. The method according to claim 36, wherein the composite score is determined with:
the level, amount or concentration of both TCL1A and AKR1C3, and
the three following clinical parameters: (i) the experience of rejection episodes before blood sampling, (ii) the recipient gender and (iii) the uptake of cyclosporine A (CsA) at blood sampling.

40. The method according to claim 36, wherein subclinical kidney rejection is subclinical T-cell mediated kidney rejection (sTCMR), subclinical antibody-mediated kidney rejection (sABMR), and/or mixed sTCMR/sABMR.

41. The method according to claim 30, wherein step 1) comprises:
a) determining a composite score, with:
the level, amount or concentration of the at least one biomarker selected from the group consisting of TCL1A and AKR1C3; and
one, two, three or four clinical parameters selected among:
the experience of rejection episodes before blood sampling,
the recipient gender,
the allograft rank, and
the number of donor-recipient HLA mismatches,
wherein said composite score is established using Formula (3):
$$composite score = \sum ( {β}_{previous rejection episode} \times previous rejection episode + {β}_{allograft rank} \times allograft rank + {β}_{HLA mismatches} \times HLA mismatches + {β}_{recipient gender} \times recipient gender + {β}_{TCL 1 A} \times Expr ( TCL 1 A ) + {β}_{AKR 1 C 3} \times Expr ( AKR 1 C 3 ) ) + {β}_{0} \qquad ( 3 )$$
wherein:
“β_{TCL1A}”, “β_{AKR1C3}”, “β_{previous rejection episode}”, “β_{allograft rank}”, “β_{HLA mismatches}”, and “β_{recipient gender}” represent the regression coefficients for each predictor among the level, amount or concentration of the biomarkers and the clinical parameters;
“previous rejection episode” represents the predictor variable defining the experience of rejection episodes before blood sampling, with 0=“no previous rejection episodes” and 1=“one or several previous rejection episodes”;
“allograft rank” represents the predictor variable defining the occurrence of previous transplantation, with 0=“no previous transplantation” and 1=“one or several previous transplantations”;
“HLA mismatches” represents the predictor variable defining the occurrence of donor-recipient HLA mismatches, with 0=“3 or less HLA-A, -B and/or -DR mismatches”, and 1=“strictly more than 3 HLA-A, -B and/or -DR mismatches”;
“recipient gender” represents the predictor variable defining the gender of the graft recipient, with 0=“female” and 1=“male”;
“Expr(TCL1A)” and “Expr(AKR1C3)” represent the predictor variables defining the level, amount or concentration of TCL1A and AKR1C3, respectively; and
“β₀” represents the intercept of the equation;
b) comparing the composite score with a reference composite score determined in the at least one reference subject;
c) concluding that the subject is affected with subclinical kidney rejection when the composite score is substantially higher than the reference composite score determined in the at least one reference subject.

42. The method according to claim 41, wherein subclinical kidney rejection consists of subclinical antibody-mediated kidney rejection (sABMR).

43. The method according to claim 30, wherein the at least one reference subject is a reference population comprising two or more reference subjects.

44. The method according to claim 30, wherein said method is computed-implemented.

45. The method according to claim 30, wherein step 1) comprises a computer system for diagnosing subclinical kidney rejection in a subject, wherein said computer system comprises:
i) at least one processor, and
ii) at least one storage medium that stores at least one code readable by the processor, and which, when executed by the processor, causes the processor to:
(a) receive an input level, amount or concentration of the at least one biomarker selected from the group consisting of TCL1A and AKR1C3,
(b) analyze and transform the input level, amount or concentration to derive a composite score established using Formula (1):
$$composite score = \sum {β}_{i} {X}_{i} + {β}_{0} \qquad ( 1 )$$
wherein:
“βᵢ” represents the regression coefficient for the level, amount or concentration of each of the at least one biomarker;
“Xᵢ” represent the predictor variable for the level, amount or concentration of each of the at least one biomarker;
“β₀” represents the intercept of the equation,
(c) generate an output, wherein the output is the composite score, and
(d) provide a diagnosis of the subject as being affected or not with subclinical kidney rejection based on the output.

46. The method according to claim 30, wherein step 2) comprises:
treating the subject with an immunosuppressive therapy, and/or
treating the subject by performing surgical splenectomy, splenic embolization and/or splenic radiation of the subject's spleen.

47. A method for identifying a subject under immunosuppressive therapy as a candidate for immunosuppressive therapy weaning or minimization comprising:
1. diagnosing subclinical kidney rejection in the subject by:
a) determining the level, amount or concentration of at least one biomarker selected from the group consisting of TCL1A and AKR1C3 in a sample previously taken from the subject;
b) comparing the level, amount or concentration of the at least one biomarker with the level, amount or concentration of the same at least one biomarker determined in at least one reference subject,
wherein the at least one reference subject is:
a subject who has not undergone kidney transplantation,
a kidney transplant recipient who is not affected with subclinical kidney rejection, or
the subject investigated for subclinical kidney rejection themselves prior to kidney transplantation; and
c) concluding that the subject is affected with subclinical kidney rejection when the level, amount or concentration of the at least one biomarker is statistically significantly lower than the level, amount or concentration of the same at least one biomarker determined in the at least one reference subject, and
2) reducing or suppressing an immunosuppressive therapy in the subject if said subject is not diagnosed with subclinical kidney rejection during the first step of the method.

48. A kit-of-parts for performing step a) of the method according to claim 30, comprising means for determining the level, amount or concentration of at least one biomarker selected from the group consisting of TCL1A and AKR1C3, and optionally, means for determining the level, amount or concentration of at least one reference marker and instructions for use to perform the method.

49. The kit-of-parts according to claim 48, wherein said means are selected from the group consisting of nucleic acid probes, antibodies, and aptamers.
